(19) United States
(12) Patent Application Publication
(10) Pub. No.: US 2013/0089614 A1
(43) Pub. Date: Apr. 11, 2013

(54) Magnetic Nanoparticles and Uses Thereof

(71) Applicants: Xuefeng Zhang, Boucherville (CA); Teodor Veres, Montreal (CA)

(72) Inventors: Xuefeng Zhang, Boucherville (CA); Teodor Veres, Montreal (CA)

(21) Appl. No.: 13/704,336

(22) PCT Filed: Jun. 13, 2011
(86) PCT No.: PCT/CA11/00684

§ 371 Date: Dec. 14, 2012

Related U.S. Application Data
(60) Provisional application No. 61/354,404, filed on Jun. 14, 2010.

(51) Int. Cl. A61K 9/14

(52) U.S. Cl. CPC A61K 9/14; Y10S 977/773; Y10S 977/755; B82Y 30/00
USPC 424/489; 977/773; 977/755; 977/838

(57) ABSTRACT

Magnetic nanoparticles are provided that have a superparamagnetic core and a nanoporous silica shell surrounding the core. The shell is functionalized with amine or S-nitrosothiol groups both inside and outside the nanopores. A process to provide such nanoparticles involves hydrolyzing tetraethoxysilane (TEOS) in a microemulsion of a superparamagnetic nanoparticle to form a superparamagnetic nanoparticle encapsulated by an incompletely hydrolyzed nanoporous silica shell, and hydrolyzing an amine-containing compound or a thiol-containing compound in situ in the presence of the incompletely hydrolyzed nanoporous silica shell before hydrolysis and densification of the silica shell is complete to functionalize the nanoporous silica shell with amine or thiol groups both inside and outside the nanopores and to maintain nanoporosity of the shell. Such magnetic nanoparticles are useful as carriers for chemical or biological species, particularly for magnetic resonance imaging, optical imaging, targeted drug delivery, cell delivery and magnetic separation applications.

### CROSS-REFERENCE TO RELATED APPLICATIONS

This application claims the benefit of United States Provisional Patent Application U.S. Ser. No. 61/354,404 filed Jun. 14, 2010, the entire contents of which is herein incorporated by reference.

### FIELD OF THE INVENTION

This application relates to magnetic nanoparticles and uses thereof, particularly for drug delivery.

### BACKGROUND OF THE INVENTION

Magnetic nanoparticles have been principally studied, in the recent years, for their potential applications in a wide range of biomedical fields, such as magnetic resonance imaging, targeted drug delivery, cell delivery and magnetic separation. Currently, critical issues to be resolved are their stability and biocompatibility in circulatory system, and surface functionalizations that conjugate the targeting spacers or therapeutic agents (Xu 2007b; Fang 2009). Core/shell structures have been proposed in an effort to address the stability and biocompatibility issues, as well as to provide a template surface for the assembly of heterogeneous functions (Zhang 2007a; Stamopoulos 2008; Gupta 2005). Among all the potential candidates, silica-based shells are undoubtedly superior, due to their low cost, relatively simple synthesis, and low toxicity. Various approaches, including wet-chemistry (Ma 2006; Zhang 2008; Yi 2005; Arruebo 2007; Niu 2010), annealing (Vadalaa 2005) and arc-discharge (Zhang 2007b; Fernández-Pacheco 2006) have been developed to synthesize different types of shell morphologies. Compared to non-porous silica, nanoporous shells not only provide excellent biocompatibility but also intrinsically higher surface areas, which are especially important when employed as drug carriers (Zhao 2009a; Slowing 2007; Nguyen 2007; Nguyen 2000; Torney 2007).

Attaching functional groups onto silica shells, prior to usage, is another critical issue since they can function as linkers for a large variety of biomolecules and drugs. This is usually done by either of two main strategies, i.e., post-functionalization (Fernández-Pacheco 2006; Kang 2009) or co-condensation (Shin 2007; Hetrick 2008; Hetrick 2009). In comparison, the co-condensation reaction leads to a more homogeneous distribution of functional groups and more stable chemical conjugations.

Controllable drug release systems triggered by various external stimuli, such as pH (Xu 2007a; Casasüs 2008; Aznar 2009), enzymes (Patel 2008; Thornton 2010), antibodies (Climent 2009) and light (Nguyen 2007; Mal 2003), have been developed. In spite of some successes, currently reported systems still lack practical application in some cases, such as therapeutic detection and drug delivery and recovery (Yi 2005; Zhang 2007b; Zhao 2009b; Guerrero-Martinez 2009). Incorporation of magnetic matter into carriers by forming hybrid architectures that are manipulated by in vitro magnetic fields may be a feasible solution, and could be further used as agents for magnetic resonance imaging (MRI). For example, Fe₃O₄ nanoparticles have been used as magnetically manipulated bars and blocking caps to control the release of fluorescein molecules (Yoon 2005). Further, a supercritical antisolvent technique has been developed to produce magnetically responsive polymer/magnetite particles for targeted drug delivery (Chattopadhyay 2004).

US 2008-0045736 (Ying 2008) discloses surface functionalized nanoparticles for bioconjugation. Functional groups such as amines are coupled to the nanoparticle via silane coupling. However, the surface of these functionalized nanoparticles is only a monolayer of silane molecules with amine groups, rather than a silica shell or nanoporous silica shell structure. Therefore the functionalized nanoparticles can be only be conjugated to a monolayer of guest biomolecules on the surface of nanoparticles. This severely limits the amount of biomolecules that can be loaded on these nanoparticles.

U.S. Pat. No. 6,548,264 (Tan 2003), US 2009-0297615 (Wang 2009) and US 2004-0067503 (Tan 2004) are examples of documents that disclose the use of TEOS to form a silica shell around a magnetic nanoparticle such as Fe₃O₄. Tan 2003 further teaches that the shell can be functionalized with primary or secondary amines for conjugation to biomolecules. Wang 2009 further teaches that such coated nanoparticle may be used to deliver drugs such as doxorubicin. However, the silica shells disclosed in documents such as these are only pure, dense silica shells, rather than nanoporous silica shell. The dense silica shells are not in-situ functionalized with amine groups, they are only post-functionalized by a only monolayer of silane molecules with amine groups in a manner similar to US 2008-0045736 (Ying 2008) described previously. While drug delivery of doxorubicin is also reported, it is only possible to conjugate the drug molecules with amine groups of the outer surface of the dense silica shell. Again, this severely limits the amount of biomolecules that can be loaded on these nanoparticles.

WO 2008-005479 (Shen 2008) discloses that primary and secondary amine systems may be used in charge reversible, pH triggered, drug carrier systems and that cyclohexanedicarboxylic anhydride can be used as a linker forming amide groups. However, this document only describes the use of such a system for in conjunction with polymer, peptide and protein coatings and does not describe silica-related materials combining with amine groups.

Finally, various strategies for immobilizing biomolecules, including doxorubicin, on magnetic nanoparticles such as Fe₃O₄ are known in the art (e.g. Boyer 2010; Fu 2010).

There still remains a need in the art for a simple method of integrating a superparamagnetic core into a nanoporous silica shell, which is simultaneously functionalized in a co-condensation process, and a need for the nanoparticles produced thereby having more interesting features for practical applications such as drug delivery.

### SUMMARY OF THE INVENTION

There is provided a magnetic nanoparticle comprising: one or more cores comprising a superparamagnetic nanoparticle; and, a nanoporous silica shell surrounding the one or more cores, the shell having nanopores, the shell functionalized with amine groups both inside and outside the nanopores.

There is further provided a magnetic nanoparticle comprising: one or more cores comprising a superparamagnetic nanoparticle; and, a nanoporous silica shell surrounding the one or more cores, the shell having nanopores, the shell functionalized with thiol groups both inside and outside the nanopores.

There is further provided a process of producing an amine functionalized magnetic nanoparticle comprising: hydrolyzing tetraethoxysilane in a microemulsion of a superparamagnetic nanoparticle to form a superparamagnetic nanoparticle encapsulated by an incompletely hydrolyzed nanoporous silica shell having nanopores; and, hydrolyzing an amine-containing compound in situ in presence of the incompletely hydrolyzed nanoporous silica shell before hydrolysis and densification of the silica shell is complete to functionalize the nanoporous silica shell with amine groups both inside and outside the nanopores and to maintain nanoporosity of the shell.

There is further provided a process of producing a thiol functionalized magnetic nanoparticle comprising: hydrolyzing tetraethoxysilane in a microemulsion of a superparamagnetic nanoparticle to form a superparamagnetic nanoparticle encapsulated by an incompletely hydrolyzed nanoporous silica shell having nanopores; and, hydrolyzing a thol-containing compound in situ in presence of the incompletely hydrolyzed nanoporous silica shell before hydrolysis and densification of the silica shell is complete to functionalize the nanoporous silica shell with amine groups both inside and outside the nanopores and to maintain nanoporosity of the shell.

The core may comprise any suitable superparamagnetic nanoparticles. The superparamagnetic nanoparticles may comprise, for example, Fe₃O₄ (also known as magnetite or ferric oxide), metallic Fe, metallic Co, metallic Ni or metal alloys (e.g. FeCo, FeNi, FePt). Preferably, the superparamagnetic nanoparticles comprises Fe₃O₄. It is possible for a single nanoporous silica shell to surround one or more than one core. For example, one shell may surround one, two, three, four, five, six or more cores.

The nanoporous silica shell is functionalized with amine or thiol groups both inside and outside the nanopores. The functionalized nanoporous silica shell may be produced by co-condensing a tetraethoxysilane (TEOS) with an amine- or thiol-containing compound in presence of a microemulsion of the superparamagnetic nanoparticle to form a nanoporous core/shell structure. The superparamagnetic nanoparticle may initially comprise a coating of an organic molecule, for example, oleic acid, and a surfactant may be adsorbed on to the surface of the superparamagnetic nanoparticle to assist in subsequent formation to the nanoporous silica shell. Hydrolysis of TEOS in the presence of the superparamagnetic nanoparticle results in formation of a silica shell which forms around a superparamagnetic nanoparticle core. Initially, the silica shell is not fully hydrolyzed. However, left alone over time, the silica shell would completely hydrolyze and densify into a pure, non-porous shell of silica. It has now been found that hydrolysis of an amine- or thiol-containing compound in the presence of the incompletely hydrolyzed silica-encapsulated superparamagnetic nanoparticle before complete densification of the silica shell permits hydrolysis of the amine- or hiol-containing compound and also permits bonding of the amine- or thiol-containing compound to the silica shell resulting in amine or thiol functionalization of the nanoporous shell both inside and outside the pores with retention of a nanoporous shell structure. Use of a surfactant facilitates the formation of the silica shell, and removal the surfactant, for example by high-velocity centrifugation or ethanol washing, contributes to the retention of the nanoporous shell structure. Nanopores in the nanoporous silica shell may have average pores sizes of about 1-5 nm.

Initially in the process, a superparamagnetic nanoparticle encapsulated by an incompletely hydrolyzed nanoporous silica shell is formed. This is followed by in situ functionalization of the nanoporous silica shell by hydrolyzing the amine- or thiol-containing compound in the presence of the incompletely hydrolyzed nanoporous silica shell. There is a time interval required for the formation of the incompletely hydrolyzed nanoporous silica shell before which the amine- or thiol-containing compound is introduced. If the time interval is too long, the TEOS would be completely hydrolyzed into a dense silica shell, and cannot further react with the amine- or thiol-containing compound due to the absence of reactive groups in the silica shell. If the time interval is too short, the nanoporous core/shell structure may not be obtained. A time interval of greater than about 8 hours and less than about 30 hours is suitable to permit formation of the nanoporous core/shell structure having an incompletely hydrolyzed nanoporous silica shell. A time interval of about 24 h time is particularly suitable.

One or more amine-containing compounds may be employed. The amine-containing compound may be a primary amine, a secondary amine or a mixture thereof.

Preferably, the shell is functionalized with both primary and secondary amines. To accomplish this, separate primary and secondary amine-containing compounds may be employed, however, it is preferable to use an amine-containing compound that contains both a primary and a secondary amine group. More preferably, the shell is functionalized with equivalent amounts of primary and secondary amine groups. This can be most readily accomplished by employing an amine-containing compound comprising equal numbers of primary and secondary amine groups. The amine-containing compound also comprises a hydrolysable group that can be hydrolyzed to facilitate bonding to the silica shell. Preferably the hydrolysable group is a silane group. A most preferred example of an amine-containing compound is N-(2-aminoethyl)-3-aminopropyltrimethoxysilane (AEAP3), the use of which results in nanoporous silica shells functionalized with 2-aminoethyl-3-aminopropyl groups. One or more thiol-containing compounds may be employed. The thiol-containing compound preferably comprises a thiosilane, for example 3-mercaptopropyltrimethoxysilane. The thiol functional group in the shell may be further derivatized, for example to a S-nitrosothiol group.

Magnetic nanoparticles of the present invention comprising an amine- or thiol-functionalized nanoporous silica shell surrounding a core of a superparamagnetic nanoparticle preferably have mean diameters in a range of about 10-500 nm, more preferably about 15-200 nm, for example about 50-200 nm. The cores preferably have mean diameters in a range of about 2-25 nm, more preferably about 5-25 nm. The shell preferably has a thickness in a range of 2-100 nm, more preferably about 5-50 nm.

Magnetic nanoparticles of the present invention may be either solid or hollow. Hollow nanoparticles comprise a nanoporous silica shell having an internal volume that is not completely filled by the core, i.e. the shell has an internal diameter that is larger than the diameter of the core (or collection of cores). Within the internal volume, the core may be either free to move, or more usually the core may be bonded at a portion of the core's surface to the inner surface of shell at some location. In either case, the internal volume of the hollow nanoparticles is available for further loading by chemical or biological species. For solid nanoparticles, there is no open internal volume as the diameter of the core (or collection of cores) substantially equals the internal diameter of the nanoporous silica shell, thus, substantially the entire surface of the core is bonded to the silica shell.

An advantage of the present invention is that the silica shells may comprise a greater content of amine or thiol groups than was hitherto possible. Amine or thiol concentrations of about 1 μmol per mg of magnetic nanoparticle or greater are possible. Concentrations of up to about 1.45 μmol per mg of magnetic nanoparticle have been obtained and higher concentrations are possible. Further, it is possible to tune the content of amine or thiol groups by adjusting thickness of the nanoporous silica shell. This enhances the utility of the magnetic nanoparticles in various applications such as molecule delivery since fewer nanoparticle are required to deliver an equivalent number of molecules. The ability to tune the nanoparticle's carrying capacity based on the thickness of the nanoporous silica shell offers greater flexibility of molecule delivery design.

Magnetic nanoparticles of the present invention may be used in a wide range of applications, especially in biomedical fields. They are particularly useful as carriers for chemical or biological species, including, for example, noble metal particles, small organic or inorganic molecules, DNA, peptides or polypeptides (e.g. antibodies and other proteins), and whole cells. Applications for such carriers include, for example, magnetic resonance imaging, optical imaging, targeted drug delivery, cell delivery and magnetic separation.

In one embodiment, especially useful for drug delivery applications, the chemical or biological species may be grafted directly or indirectly to the amine groups of the nanoporous silica shells to form pH-responsive bonds. When the carrier encounters a change in pH at a site of interest, the pH-responsive bonds are broken thereby releasing the chemical or biological species at the site of interest. The pH-responsive bonds preferably comprise amide bonds between the amine groups of the nanoporous silica shell and intermediate linkers comprising a carboxylic group. The carboxylic group preferably comprises cyclohexanedicarboxylic anhydride.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 depicts (a) a self-assembly TEM image and (inset) the size distribution from more than 200 particles of Fe₃O₄/OA nanoparticles; (b) high-resolution TEM image and (inset) the fast Fourier transform pattern corresponding to the squared region; (c) XRD pattern of Fe₃O₄/OA nanoparticles; (d) TEM image of core/shell Fe₃O₄/silica nanoparticles with shells having a thickness of 56.2±0.09 nm; and, (e) TEM image of core/shell Fe₃O₄/silica nanoparticles with shells having a thickness of 63.1±0.09 nm.

FIG. 2 depicts Fe₃O₄/silica(porous) nanoparticles of the present invention, (a) TEM image and (inset) the corresponding size distribution; (b) high angle annular dark field (HAADF) image; (c) fast Fourier transform (FFTs) pattern of Fe₃O₄ core marked in (b); (d) EDS mapping; and, (e) line analysis along the axis.

FIG. 3 depicts FTIR spectra of (a) Fe₃O₄/OA nanoparticles, (b) Fe₃O₄/silica nanoparticles, and (c) Fe₃O₄/silica(porous) nanoparticles.

FIG. 4 depicts: (a) optical photograph of the reaction, separation and re-dispersion of a 2 mg/ml nanoparticles/acetone solution (A) and a 1 mg/ml fluorescamine/acetone solution (B); (b) optical photograph of the reaction, separation and re-dispersion of an acetone solution containing 50 μl AEAP3 (F) and a 1 mg/ml fluorescamine/acetone solution (G); (c) UV-Vis spectra of various concentrations of the supernatant solutions recovered after separation; and, (d) TEM image of Fe₃O₄/silica(porous) nanoparticles decorated by ultrasmall Au nanoparticles (1-2 nm).

FIG. 5 depicts hysteresis loops at 5 and 300 K of: (a) Fe₃O₄/OA, where the upper insert is an enlargement of the graph near the origin; (b) Fe₃O₄/silica and Fe₃O₄/silica(porous) nanoparticles after field cooling; (c) ZFC-FC magnetization curves of (i) Fe₃O₄/OA, (ii) Fe₃O₄/silica and (iii) Fe₃O₄/silica(porous) nanoparticles under an applied magnetic field of 50 Oe; (d) scheme of the interaction between two magnetic nanoparticles as distances; and, (e) The photographs of Fe₃O₄/silica(porous) nanoparticles dispersed in water, with and without magnetic separation.

FIG. 6 depicts a scheme showing coupling of doxorubicin (DOX) molecules with primary and secondary amine groups of silica shells using 1,2-cyclohexanedicarboxylic anhydride (CA) as linkers.

FIG. 7 depicts: (a) normalized UV-Vis absorption spectra of doxorubicin molecules for separated supernatant solutions at various releasing times and pH; and, (b) release profiles of doxorubicin molecules from Fe₃O₄/silica(porous) nanoparticles in buffer solutions of pH 5.0, 6.0 and 7.4.

FIG. 8 depicts: (a) release profiles of doxorubicin molecules from Fe₃O₄/silica(porous) nanoparticles as a function of time in buffer solutions of pH 5.0, 6.0 and 7.4; and, (b) correlation between the release rate constant (k_{H}) and pH.

FIG. 9 depicts Fe₃O₄/OA nanoparticles: (a) a self-assembly TEM image with (inset) the corresponding size distribution; and, (b) a high-resolution TEM image with (inset) the corresponding fast Fourier transform pattern from the central region.

FIG. 10 depicts: (a) TEM image of Fe₃O₄/silica nanoparticles; (b) TEM image of Fe₃O₄/silica(H) nanoparticles; (c) a high angle annular dark field (HAADF) of Fe₃O₄/silica(H) nanoparticles; and, (d) statistical size distributions.

FIG. 11 depicts: (a) TEM and the HRTEM (inset) images of Fe₃O₄/silica(H) nanoparticles; and, (b) EDS analysis along the axis of Fe₃O₄/silica(H) nanoparticles.

FIG. 12 depicts XRD patterns of: (a) the standard card of Fe₃O₄ powders (JCPDS 880315); (b) Fe₃O₄/OA nanoparticles; (c) Fe₃O₄/silica nanoparticles; and, (d): Fe₃O₄/silica(H) nanoparticles.

FIG. 13 depicts FTIR spectra of: (a) Fe₃O₄/silica(H) nanoparticles; (b) Fe₃O₄/OA nanoparticles; and, (c) Fe₃O₄/silica nanoparticles.

FIG. 14 depicts: (a) hysteresis loops at 50 K and 300 K of Fe₃O₄/OA nanoparticles; (b) hysteresis loops at 50 K and 300 K of Fe₃O₄/silica and Fe₃O₄/silica(H) nanoparticles; (c) corresponding magnification of FIG. 14(a) near the origin; and, (d) corresponding magnification of FIG. 14(b) near the origin, where the inset in FIG. 14(d) shows the ZFC-FC magnetization curve of Fe₃O₄/silica(H) nanoparticles under an applied magnetic field of 50 Oe.

FIG. 15 depicts: (a) a schematic diagram for fluorescein release process (upper), and photographs (below) for as-made samples (A), magnetically-separated samples (B), and C-G: magnetically-separated samples (C-G) after release times of 2, 4, 6, 8, 10 and 12 hours; and, (b) a graph depicting fluorescein concentration variation as a function of releasing time at room temp and at 37° C., with the inset showing the corresponding fluorecence spectra at 37° C.

FIG. 16 depicts a conjugation and release scheme of doxorubicin molecules with secondary amine groups of Fe₃O₄/silica(H) nanoparticles using 1,2-cyclohexanedicarboxylic anhydride as a linker.

FIG. 17 depicts normalized UV-Vis absorption spectra of doxorubicin molecules for separated supernatant solutions after various loading times, where the inset depicts the loading profile of doxorubicin molecules as a function of time.

FIG. 18 depicts release profiles of doxorubicin molecules from Fe₃O₄/silica(H) nanoparticles based on UV absorption as a function of time in buffer solutions of pH 5.0, 6.0 and 7.4, where FIG. 18(a) and FIG. 18(b) are at room temperature and FIG. 18(c) and FIG. 18(d) are at 37° C.

FIG. 19 is a graph depicting correlation between release rate constant (k_{H}) and pH for the release of doxorubicin molecules from Fe₃O₄/silica(H) nanoparticles at room temperature and 37° C.

FIG. 20 is a scheme depicting nitric oxide grafting and release, showing protocols that transform —SH functional groups to —SNO groups of Fe₃O₄/silica(SH) NPs by reacting the —SH functional groups with t-butyl nitrite or NaNO₂ to form —SNO groups.

FIG. 21 depicts TEM images of (a) hollow silica (SNO) nanoparticles and (b) a magnified image of (a), where SNO groups are formed by HCl+NaNO₂.

FIG. 22 depicts TEM images of (a) ultrathin gold nanoparticles, (b) core/shell Fe₃O₄/Silica (SNO) nanoparticles, (c) a magnified image of (b), and (d) ultrathin gold nanoparticles-coupled Fe₃O₄/silica (SNO) nanoparticles, where SNO groups are formed by t-butyl nitrite.

FIG. 23 depicts FTIR spectra of: (a) Fe₃O₄/silica(H) NPs; (b) Fe₃O₄/silica(—SH) NPs; (c) Fe₃O₄/silica(—SNO) NPs; and Fe₃O₄/silica(—SNO) NPs after NO release.

FIG. 24 depicts a graph showing the quantitative evaluation of NO release of 1.5 mg Fe₃O₄/silica(—SNO) NPs in 20 ml PBS 7.2 buffer.

### DESCRIPTION OF PREFERRED EMBODIMENTS

### Materials and Methods

All reagents referred to herein are commercially available. Oleic acid (OA, 90%), anhydrous 1-hexanol (99%), octyl ether (98%), ammonia solution (NH₄OH, 28-30 wt % in water), Triton™ X-100, hexane (95%), cylcohexane (99.5%), dimethyl sulfoxide (DMSO, 99%), 1,2-cis-cyclohexanedicarboxylic anhydride (98%), triethylamine (98%), tetraethoxysilane (TEOS, 99.999%) sodium hydroxide (99%), tetrachloroaurate(III) hydrate (99.99%), and doxorubicin hydrochloride (98%) were purchased from Sigma-Aldrich Inc. Iron pentacarbonyl (99.5%) was purchased from Strem Chemicals, Inc. (Newburyport, Mass.). N-(2-aminoethyl)-3-aminopropyltrimethoxysilane (AEAP3, 90%) was purchased from Gelest (Tullytown, Pa.). N-(trimethoxysilylpropyl)polyethylenimine (PS076, 50%) was purchased from UCT Specialties, LLC). Fluorescamine was purchased from MP Biomedicals, LLC.

The size and morphology of nanoparticles were analyzed using a Hitachi S-4700 transmission electron microscopy (TEM) operated at a voltage of 30 kV. Microstructure and composition of the samples were characterized by using a high resolution TEM (HRTEM), selected area electron diffraction (SAED), and energy dispersive X-ray spectroscopy (EDS) on a JEOL 2010F (200 kV) transmission electron microscopy. TEM samples were prepared by dropping 25 μl of particle dispersion in hexane on amorphous carbon coated copper grids, and drying under vacuum over the night. FTIR spectra were collected with a Nicolet Fourier spectrophotometer at wave numbers between 600 cm⁻¹ and 4000 cm⁻¹. Siemens D-500 X-ray diffractometer with CuKa (λ=0.154 nm) radiation at a voltage of 30 kV and a current of 30 mA was used to study the phase structure of the nanoparticles at a scan step of 0.2°. UV-Vis spectra were collected on a Perkin Elmer Lamda 950 spectrometer. Magnetic measurements of major hysteresis loops (MHL) at different temperatures as well as zero-field cooled (ZFC) magnetization processes were performed with a Quantum Design PPMS model 6000 magnetometer. A Thermo Scientific NanoDrop™ 3300 fluorospectrometer was employed to detect the concentration variation of fluorescein molecules.

The theoretical model employed to fit the temperature-dependent zero-field-cooled (ZFC) magnetization processes is described in detail in Sappey 1997 and is defined by the function:

$$\begin{matrix}{m}_{ZFC} ( T ) = \frac{{μ}_{0} {M}_{s}^{2}}{3 {K}_{eff}} {HV}_{tot} W ( {D}_{b} , ∞ , 3 ; D ) + \frac{{μ}_{0} π {M}_{s}^{2}}{18 {k}_{B} T} {V}_{tot} W ( 0 , {D}_{b} , 6 ; D ) \\ with & ( 1 ) \\ W ( {D}_{1} , {D}_{2} , k ; d ) = \frac{{\int }_{{D}_{1}}^{{D}_{2}} {D}^{k} f ( D ) d D}{{\int }_{0}^{∞} {D}^{3} f ( D ) d D} & ( 2 )\end{matrix}$$

where μ₀ is the vacuum absolute permeability, Mₛ the saturation magnetization, H the external applied field, K_{eff} the anisotropy constant, Vₜₒₜ the total magnetic volume of the sample and f(D) the lognormal probability density distribution of the NP diameters. D_{b} is the deblocking diameter and relates the blocking temperature T_{b} to the blocking volume V_{b} of particles via Eq. (3):

$${T}_{b } = \frac{U ( H )}{{k}_{B} ln \frac{{τ}_{m}}{{τ}_{0}}} \qquad ( 3 )$$

In Eq. (3), U(H)=K_{eff}V(1−H/H_{C})^{α} is the energy barrier of a given particle of volume V and coercive field H_{C}. k_{B} is the Boltzmann constant whereas τₘ and τ₀ are the “experiment time” and the lattice vibration period, respectively (Dormann 1997). α is a phenomenological constant of value 1.5 and it is related to the field dependence of the magnetic energy barrier (Cullity 1972). For the interacting model, the transformation of the Langevin function argument

$$\frac{μ H}{{k}_{B} T} -> \frac{μ H}{{k}_{B} ( T + {T}^{*} )} \qquad ( 4 )$$

as described in Vargas 2005 and Allia 2001 has been taken into consideration.

### Examples 1-8

### Solid Nanoporous Magnetic Nanoparticles Functionalized with Primary and Secondary Amines

In Examples 1-8 a co-condensation synthesis and subsequent characterization is described in connection with solid superparamagnetic core/shell Fe₃O₄/silica(porous) nanoparticles containing both primary and secondary amine groups in the same nanoporous silica shell. Both the primary and secondary amine groups of the nanoporous shells not only can be used for optical labeling, either by direct conjugation with fluorescent molecules or by coupling with plasmonic Au nanoparticles, but can also be used for pH-regulated drug delivery. Fe₃O₄/silica(porous) nanoparticles functionalized with 1,2-cyclohexanedicarboxylic anhydride as click linkers provide considerable ability to couple with doxorubicin molecules via amides. Moreover, the coupled doxorubicin molecules are relatively stable at neutral pH 7.4, but can be rapidly released in the range of pH 5.0 to 6.0 due to the hydrolysis of amide bonds under assistances of neighboring carboxylic acid groups. Combined with the magnetic nature of the Fe₃O₄ cores, these functionalities present a multifunctional nanoparticle that can be used for both magnetically-targeted drug delivery while providing the possibility of multimodal imaging, using both optical and MRI techniques.

Thus, solid superparamagnetic amino-functionalized Fe₃O₄/silica(porous) core/shell nanoparticles with nanoporous shells were developed by a wet-chemical method. These nanoparticles have a mean diameter of about 65 nm, having a 15.1 nm Fe₃O₄ core and a nanoporous shell. Such nanoparticles have a magnetic anisotropy of (1.15±0.05)×10⁴ J/m³ and a saturated magnetization of 1.1 emu/g. Based on a theoretical model, the temperature-dependent magnetization processes point toward a topology-dependent weakened interaction between superparamagnetic Fe₃O₄ cores due to the steric hindrance of the shells, contributing to a non-interacting dispersibility in aqueous media. The nanoporous silica shells contain an equivalent amount of both primary and secondary amine groups up to a concentration of 1.45 μmol mg⁻¹, and exhibit a significant feature for drug delivery. Doxorubicin, as one of the most widely used anti-cancer drugs, was coupled to the nanoporous silica shells by pH-responsive amide groups. It is found that under low pH conditions such as 5 to 6, the doxorubicin molecules can be effectively released, while at pH 7.4 they are relatively stable. The greatest extent of release of doxorubicin was about 9.8 mg for 100 mg Fe₃O₄/silica(porous) nanoparticles at pH 5 after 63 hrs, with 76% effectively released after 10 hrs. At pH 7.4, only 3.8% and 9% were released after 10 hrs and 63 hrs, respectively. These properties demonstrate that Fe₃O₄/silica(porous) nanoparticles are very suitable for magnetically-targeted drug delivery.

### Example 1

### Synthesis of Fe₃O₄ nanoparticles

Oleic acid-coated Fe₃O₄ (Fe₃O₄/OA) nanoparticles were synthesized based on a well-known process (Woo 2004). Under a nitrogen flow, a mixture of 20 ml octyl ether and 1.92 mL oleylamine was mixed at room temperature for about 10 minutes. This solution was subsequently heated to 100° C. in 20 min, remaining nearly colorless. At 100° C., 0.4 ml of iron pentacarbonyl were quickly injected into the solution under a fast argon flow, and the temperature was raised to 290° C., at a rate of 2° C./min. The solution was refluxed at 290° C. for 2 hours and cooled down to room temperature by removing the heating source. During the reflux process, the solution experienced a color change from light yellow, to colorless to black. The resultant product of 15 nm Fe₃O₄/OA nanoparticles was precipitated by adding excess anhydrous ethanol, and separated by centrifugation (9000 rpm). The product purified at least three times was dried under vacuum, and then kept in vacuum for a long-term storage.

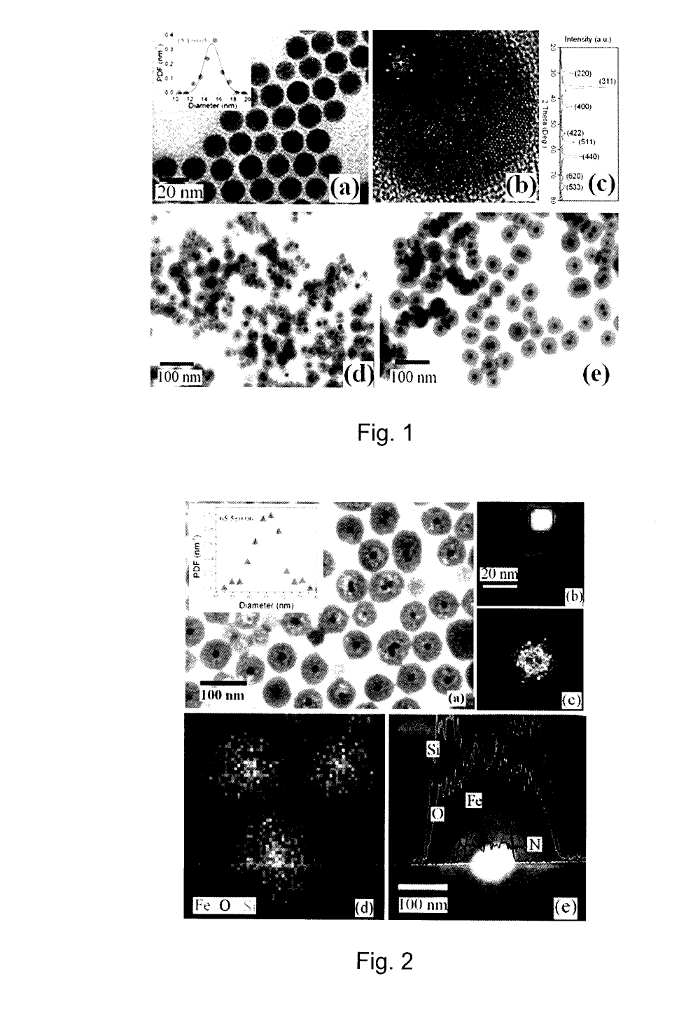

FIG. 1 (a) shows a typical transmission electron microscopy (TEM) image of as-synthesized Fe₃O₄/OA (OA=oleic acid) nanoparticles. The particles are seen to have a narrow size distribution and form a self-assemble super-lattice. The measurement of about 200 particles has shown that the particles are essentially spherical in shape, with a mean diameter of 15.1 nm. FIG. 1(b) shows a high-resolution TEM image and its corresponding fast Fourier transform (FFT) pattern. The FFT pattern, obtained from a large region at the center-right of FIG. 1(b), has a symmetrical lattice, indicating the single crystalline nature of the nanoparticles. FIG. 1(c) shows an XRD pattern of the Fe₃O₄/OA nanoparticles. The positions and relative intensities of all diffraction peaks match those obtained on standard Fe₃O₄ powders, clearly confirming that the product is Fe₃O₄ rather than other iron oxides. The calculation for the lattice spacing of the (311) plane gives a=0.8462 nm, which is 0.77% larger than that of the bulk Fe₃O₄ (0.8397 nm). The origin of anomalous lattice expansion at the nanoscale may be attributed to the decrease of the electrostatic forces caused by the valence reduction of the Fe ions (Tsunekawa 2000). The mean grain size was estimated to be 13.8 nm according to Scherrer's formula (Warren 1969): Dₕₖₗ*=Kλ/(B cos θ), in which K=0.9, λ=0.154046 nm, B is the width at half-height of the (311) peak, and 8 is the angle corresponding to the (311) peak. The statistical grain sizes by both methods are in basically agreement, further implying that each individual particle is a single crystal.

### Example 2

### Synthesis of Core/Shell Fe₃O₄/Silica Nanoparticles

Non-porous core/shell Fe₃O₄/silica nanoparticles were fabricated by hydrolyzing TEOS in a water-in-oil microemulsion that contains the Fe₃O₄/OA nanoparticles from Example 1 as seeds. Briefly, Fe₃O₄/OA nanoparticles were first dispersed in cyclohexane, at a concentration of 1 mg/mL, and then 0.5 ml of the Fe₃O₄-containing cyclohexane dispersion were rapidly injected into a mixture of 1.77 g of Triton™ X-100, 1.6 ml of anhydrous 1-hexanol and 7 ml of cyclohexane under a strong vortex for about 1 h. Subsequently, 0.5 mL of ammonia solution (28-30% ammonia solution to water in a 1:4 ratio by volume) were added in the above solution and shaken for another 1 h. Finally, 25 μl of TEOS were added, and the mixture was allowed to react for 24 h. To further increase the thickness of the silica shells, an additional 25 μl of TEOS were added and left for another 24 h under the same conditions. Two kinds of Fe₃O₄/silica nanoparticles were prepared, with silica shell thicknesses of about 20 and 24 nm, by adding 25 and 25+25 μl of TEOS. These two types are denoted as Fe₃O₄/silica(1) and Fe₃O₄/silica(2), respectively. The as-fabricated products were separated by centrifugation at 9000 rpm, washed with ethanol, and the centrifugation/wash procedure was repeated three times. The resultant nanoparticles were dried under vacuum, or directly dispersed in de-ionized water for characterization.

Silica-coated core/shell nanoparticles by using hydrophobic Fe₃O₄/OA nanoparticles as precursors have been reported previously (Zhang 2008; Qian 2009; Santra 2001). By adjusting the TEOS contents such as 25 μl and 25+25 μl (“+”=a reaction time interval of 24 h), two different thicknesses of silica shells were present, i.e. Fe₃O₄/silica(1)-(2). As the concentration of TEOS increased during with a reaction time interval of 24 h, the silica shells became thicker, as shown in FIGS. 1 (d) and (e). The statistic analysis of each sample shows that the thickness of the shells can be stably increased, obtaining total particle diameters of 56.2±0.09 nm and 63.1±0.09 nm. It should be noted that there are no obvious interfaces between silica shells for the Fe₃O₄/silica(2), indicating a continuous growth of separate TEOS condensation processes.

### Example 3

### Synthesis of Core/Shell Fe₃O₄/Silica(Porous) Nanoparticles

Magnetic nanoparticles in accordance with the present invention having Fe₃O₄ cores and porous silica shells with equivalent amounts of primary and secondary amines (denoted Fe₃O₄/silica(porous) nanoparticles) were synthesized in a two-step procedure by hydrolyzing TEOS and AEAP3 molecules. The first step comprised the synthesis of Fe₃O₄/silica(1) nanoparticles of Example 2 by hydrolyzing TEOS. After forming silica shells, 25 μl of AEAP3 were injected into the reaction mixture for another 24 h. The resultant product was denoted as Fe₃O₄/silica(porous) nanoparticles. The products were centrifuged at 9000 rpm and washed with anhydrous ethanol three times, and finally dispersed in de-ionized water for use.

FIG. 2 (a), and its inset, show a TEM image and the corresponding particle size distribution of Fe₃O₄/silica(porous) nanoparticles. The as-synthesized nanoparticles are all spherical in shape with an average total diameter of 65.5±0.06 nm, which is basically in agreement with what was found for Fe₃O₄/silica nanoparticles of Example 2. Compared with the structural features of Fe₃O₄/silica nanoparticles, it is worth noting that the particles of Example 3 present nanoporous structures with sponge-like ultra-thin pores. Moreover, ultra-thin pores with bicontinuous channels extend to the NP surface, as shown in FIG. 2 (b). Such a feature offers a distinct advantage for drug storage and delivery. Additionally, some smaller hollow and porous silica nanoparticles appear in the product. Energy dispersive X-ray spectroscopy (EDS) analyses shown in FIGS. 2 (d) and (e) reveal the elemental distribution of iron, oxygen, silicon and nitrogen. One observes a core/shell feature, obviously indicating that the core is rich in iron and oxygen, while the shell is mainly made of silicon, oxygen and nitrogen. The FFTs pattern in FIG. 2 (c), corresponding to the squared region in FIG. 2 (b), confirms that the Fe₃O₄ core is still monocrystalline.

Without being held to any particular mechanism of action, based on obtained results, it appears that in the W/O type micro-emulsion system, Triton™ X-100 molecules replace the OA molecules and take them into the water phase. This results in an aqueous reaction cell for the condensation and growth of TEOS molecules on the surface of hydrophobic Fe₃O₄ nanoparticles. It should be noted that in the reaction process, the Triton™ X-100 molecules also played another role in limiting the TEOS condensate to a non-porous shell because they were strongly adsorbed on the surface of Fe₃O₄ nanoparticles by polyethylene oxide groups. Subsequently, the silica shell can further react with AEAP3 molecules, forming a mixed region comprising a complex of hydrolyzed silica shells and incompletely hydrolyzed TEOS and AEAP3, as well as adsorbed Triton™ X-100 molecules. Ultra-thin pores were formed due to steric hindrance of —O₂Si(OH)R and —O₃SiR backbones (R represents an aminoethylaminopropyl group), and finally retained after removing the Triton™ X-100 molecules by ethanol washing. The co-effect of long molecule backbones and surfactant (Triton™ X-100) is important in the formation of nanoporous silica shells.

### Example 4

### Comparison of Surface Chemistry of Fe₃O₄/Silica(Porous) Nanoparticles to Fe₃O₄/OA Nanoparticles and Fe₃O₄/Silica Nanoparticles

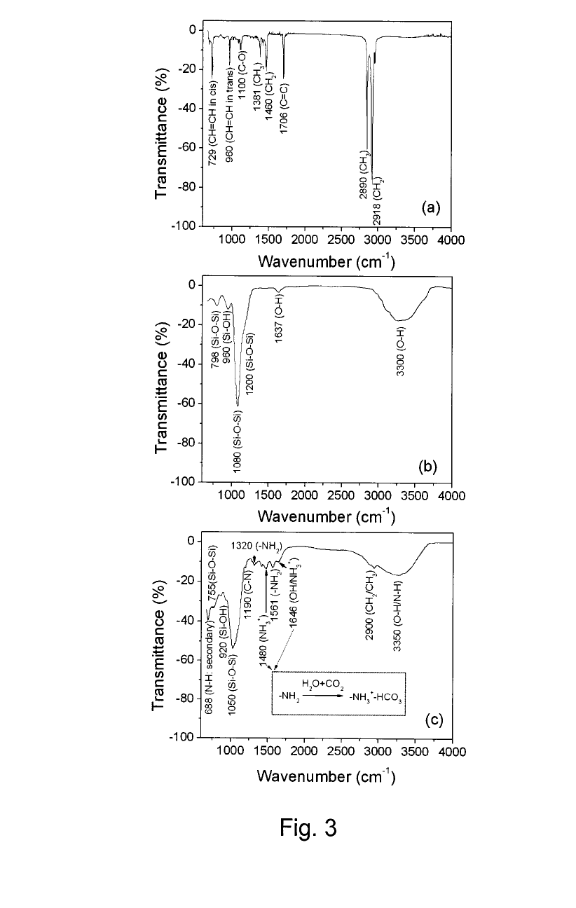

In order to confirm the functional groups on the surface of nanoparticles, Fourier transmission infrared (FTIR) spectra were collected on (a): Fe₃O₄/OA, (b): Fe₃O₄/silica and (c): Fe₃O₄/silica(porous) nanoparticles, as shown in FIG. 3. The absence of —OH vibrations at about 3300 cm⁻¹ from acid groups indicates that all the OA molecules have reacted with the Fe₃O₄ surface and no physi-sorbed oleic acid molecules remained. This results in a self-assembled oleate monolayer around the surface of Fe₃O₄ nanoparticles. Thus the Fe₃O₄/OA nanoparticles bear —CH₃ groups at the free termini of OA chains, leading to a hydrophobic behavior. As shown in FIG. 3(b), asymmetric and symmetric stretching vibrations of ≡Si—O—S≡ were observed at about 1080 cm⁻¹ and 798 cm⁻¹, respectively, corresponding to characteristic peaks of silica (Simons 1978; Coluccla 1978). The peaks at 3400 cm⁻¹, 1637 cm⁻¹ and 960 cm⁻¹ are assigned, respectively, to the stretching and deformation vibrations of adsorbed water molecules and the stretching mode Si—OH (hydroxyl groups) (Back 1991; Chukin 1977; Bertoluzza 1982). The FTIR spectrum of Fe₃O₄/silica(porous) nanoparticles is shown in FIG. 3(c). The characteristic peaks of silica at 1050 cm⁻¹ (asymmetric stretching ≡Si—O—Si≡), 920 cm⁻¹ (symmetric streching ≡Si—O—Si≡) and 755 cm⁻¹ (Si—OH), shifted to lower wavenumbers compared to that of Fe₃O₄/silica nanoparticles, may be ascribed to the effect of aminoethylaminopropyl groups. The broad peak at about 3350 cm⁻¹ is due to an overlap of hydrogen-bonded O—H and N—H stretching. The peaks at about 2900 cm⁻¹ are due to stretching vibrations of —CH₂— bonds. The peaks at 1320 cm⁻¹ and 1561 cm⁻¹ are consequences of the vibrations of primary amine groups (—NH₂), while the peak at 688 cm⁻¹ results from the vibrations of secondary amine groups (Culler 1984). However, the peaks at 1480 cm⁻¹ and 1646 cm⁻¹ indicate that the primary amine groups partly reacted with carbon dioxide and water when AEAP3 molecules were hydrolyzed in air, and form a resin containing —NH₃⁺—HCO₃. This bicarbonate salt can be easily removed by heating at about 80° C. (Culler 1984). The FTIR analyses confirmed that the silica shells of Fe₃O₄/silica(porous) nanoparticles are functionalized by hydroxyls, primary and secondary amine groups.

### Example 5

### Determination of Amine Content of Fe₃O₄/Silica(Porous) Nanoparticles

The amine group concentrations on the Fe₃O₄/silica(porous) nanoparticles were determined using a fluorescamine test. Fluorescamine is non-fluorescent but rapidly reacts with primary amine groups to form a fluorescent product that fluoresces at 465-475 nm, so it has become a common method to measure the quantity of primary amine groups in many assays (Udenfriend 1972). This approach to measure the content of amine groups can be reasonably expected to give a proper evaluation of bioconjugation ability of Fe₃O₄/silica(porous) nanoparticles.

In brief, Fe₃O₄/silica(porous) nanoparticles with various loadings were dispersed in 1 ml solutions of fluorescamine in acetone. Subsequently, the dispersions containing the nanoparticles were centrifuged at 9000 rpm for 10 min, and the supernatant solution fluorescence was evaluated by UV-Vis spectroscopy. As a control experiment, various concentrations of fluorescamine/acetone solutions were processed by the same method. All the tests were repeated at least five times.

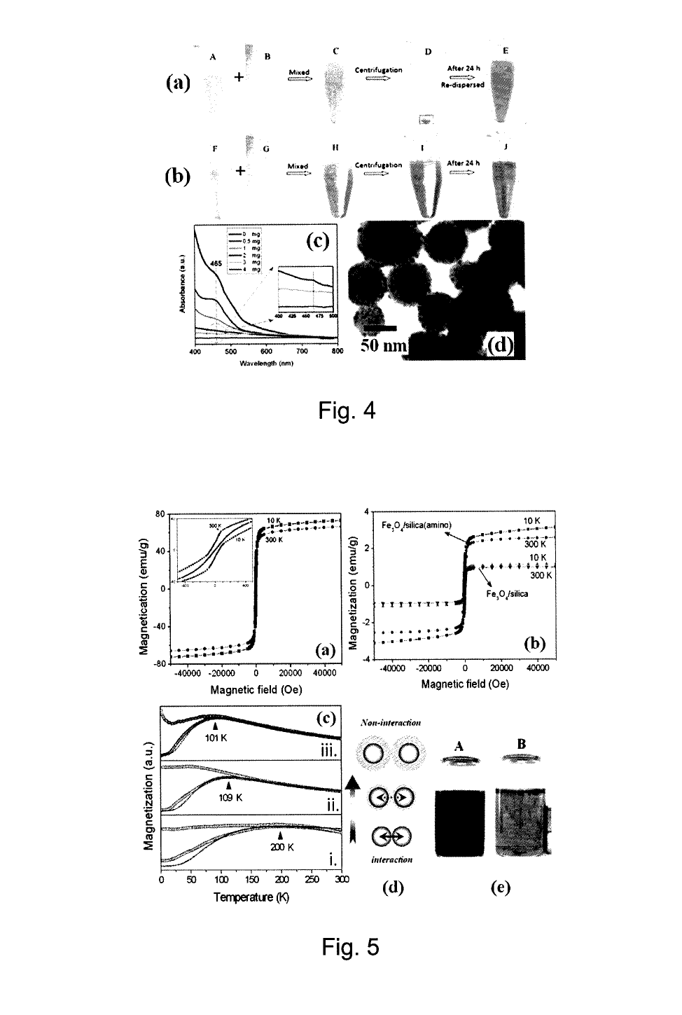

FIGS. 4 (a) and (b) show the reaction scheme. Firstly, a 2 mg/ml nanoparticle/acetone solution and a 1 mg/ml fluorescamine/acetone solution were prepared, and then mixed, using a ratio of 1:1, followed by slight shaking for one minute. When the reaction was complete, the color of the mixture changed from light gray to yellow, C of FIG. 4(b). By centrifuging the mixture at 9000 rpm for ten minutes, a clear supernatant (D) was obtained. After one day, the separated nanoparticles could be redispersed ultrasonically, with a deep yellow color. Under the same experimental conditions, 50 μl AEAP3 molecules were dissolved in a 1 ml solution of 1 mg/ml fluorescamine/acetone, and subjected to the same centrifugation process, as shown in FIG. 4(b). The color of the mixture was similar to that of the Fe₃O₄/silica(porous) nanoparticles, but was difficult to separate by centrifugation, even over a longer time. The florescamine labeling test confirms the existence of primary amine groups in the Fe₃O₄/silica(porous) nanoparticles. To further evaluate the content of amine groups, UV-V is spectra (FIG. 4(c)) were used to monitor the change of the concentration of fluorescamine molecules in solution. The quantity of amine groups in 1 mg Fe₃O₄/silica(porous) nanoparticles is about 1.45 μmol.

### Example 6

### Decoration of Fe₃O₄/Silica(Porous) Nanoparticles by Ultrasmall Au Nanoparticles

Ultrasmall gold nanoparticles used to decorate the surface of the Fe₃O₄/silica(porous) nanoparticles were synthesized by the Duff's method (Duff 1993a; Duff 1993b). Thus, deionized water (45 ml), NaOH (1 M, 0.3 mL) and a THPC solution (12 μl) were first mixed. Under vigorous stirring, a solution of hydrogen tetrachloroaurate(III) hydrate (25 mM, 2 mL) was then injected resulting in a rapid formation of a dark orange-brown solution. The solution were aged overnight at about 0° C. The resultant ultrasmall Au solution was mixed with 1 mg Fe₃O₄/silica(porous) nanoparticles for overnight and subsequently separated by a permanent magnet. Au-decorated Fe₃O₄/silica(porous) nanoparticles were washed for three times and dispersed in deionized water for characterization.

Amino-functionalized silica shells, with nanoporous structures, not only exhibit a big loading capacity for guest molecules, but also a fast immobilization to ultrasmall gold nanoparticles by gold-amine interactions (Westcott 1998). A TEM image in FIG. 4(d) clearly shows that the gold nanoparticles can be attached on the surface of Fe₃O₄/silica(porous) nanoparticles. UV-Vis. spectra were carried out to confirm that the Au-decorated Fe₃O₄/silica(porous) nanoparticles display a plasmonic resonance absorption at wavelength of about 500 nm.

### Example 7

### Magnetic Properties of Fe₃O₄/Silica(Porous) Nanoparticles

FIGS. 5(a) and 5(b) show the major hysteresis loops (MHLs) and corresponding enlargements of Fe₃O₄/OA, Fe₃O₄/silica and Fe₃O₄/silica(porous) nanoparticles at 10 K and 300 K. The as-synthesized Fe₃O₄/OA nanoparticles, in FIG. 5(a), exhibit typical superparamagnetic behavior, consisting of Langevin-type curves of nearly zero coercive fields at room temperature. As expected, in the low temperature regime, blocked (ferromagnetic) particles become preponderant and the MHLs become slightly hysteretic, with an increased saturation magnetization (Ms) of about 73 emu/g and a coercive field (H_{C}) of about 53 Oe. Chemical analysis result from Guelph Chemical Laboratories Ltd. shows that the as-synthesized Fe₃O₄/OA nanoparticles contain about 62.5 wt % iron, corresponding to 86.3 wt % Fe₃O₄ and 13.7 wt % OA regardless of other impurities. This corresponds to a net value of 84.5 emu/g for pure Fe₃O₄ nanoparticles, as high as the result reported in Sun et al. (Sun 2004). However, the organic species can be removed completely on silica coating, as seen from the FTIR spectra. As shown in FIG. 5(b), the Ms values of Fe₃O₄/silica and Fe₃O₄/silica(porous) nanoparticles are about 3.1 and about 1.1 emu/g, corresponding to the nomagnetic silica compositions of 96.3 wt % and 98.7 wt %, respectively. All recorded MHLs present a decrease of both coercive field and saturation magnetization with the temperature and the obtained experimental variations are typical for iron-based nanoparticles (Vargas 2005).

FIG. 5(c) shows the temperature-dependent zero-field-cooling (ZFC) and field-cooling (FC) magnetization curves of Fe₃O₄/OA, Fe₃O₄/silica and Fe₃O₄/silica(porous) nanoparticles, respectively, measured at an applied magnetic field of 50 Oe. The ZFC curve of Fe₃O₄/OA nanoparticles exhibits a broader maximum of about 200 K, which is taken as Tₘₐₓ, and an irreversibly branching temperature at 279 K (T_{Br}). The Tₘₐₓ values of Fe₃O₄/silica and Fe₃O₄/silica(porous) nanoparticles become more obvious and shift to lower temperatures at 109 K and 101 K, respectively, as the thickness of shells increase, although the Fe₃O₄ cores were not changed. For isolated, non-interacting nanoparticles, Tₘₐₓ is normally related to the blocking temperature (T_{B}) at which the particles undergo a phase transition from ferromagnetic to superparamagnetic. As for the ZFC analysis, the experimental curves were compared to a theoretical model based on the blocking behavior of assemblies of superparamagnetic nanoparticles (Sappey 1997). The mutual interactions between nanoparticles were accounted for by using the “T*” formalism (Vargas 2005; Allia 2001) that consists of adding a fictional “interacting” temperature to the actual temperature in the denominator of the Langevin function argument. According to this formalism, larger values of T* indicate stronger interactions between particles. The most dilute sample, Fe₃O₄/silica(porous) nanoparticles, was assumed as “non-interacting” and T* was accordingly set to zero. σ_{D} and K_{eff}, fit parameters in the theoretical model, are described previously. The experimental measurements are well reproduced theoretically for Fe₃O₄/silica and Fe₃O₄/silica(porous) nanoparticles. Standard deviations of size distribution and magnetic anisotropy constant were estimated at σ_{D}=0.18±0.01 and K_{eff}=(1.15±0.05)×10⁴ J/m3, respectively, with acceptable precision, in agreement with that of bulk Fe₃O₄ (1.1×10⁴ J/m³ to 1.3×10⁴ J/m³) (Cullity 1972). As expected, mutual interactions among particles increase as the particles become closer packed. In terms of ZFC behavior, the peak of these curves is shifted to higher temperatures as the reciprocal distances between particles get smaller, as shown in FIG. 5(d). We identify numerical values of T*=30±8 K for Fe₃O₄/silica and 550±210 K for Fe₃O₄/OA, respectively. The “interacting” temperature in the Fe₃O₄/OA nanoparticles is larger since the particles are closer to each other. Lower blocking temperature for the Fe₃O₄/silica(porous) nanoparticles, arising from the steric hindrance of the silica shells, provide a noninteracting monodispersibility.

### Example 8

### pH-Regulated Doxorubicin Release from Fe₃O₄/Silica(Porous) Nanoparticles

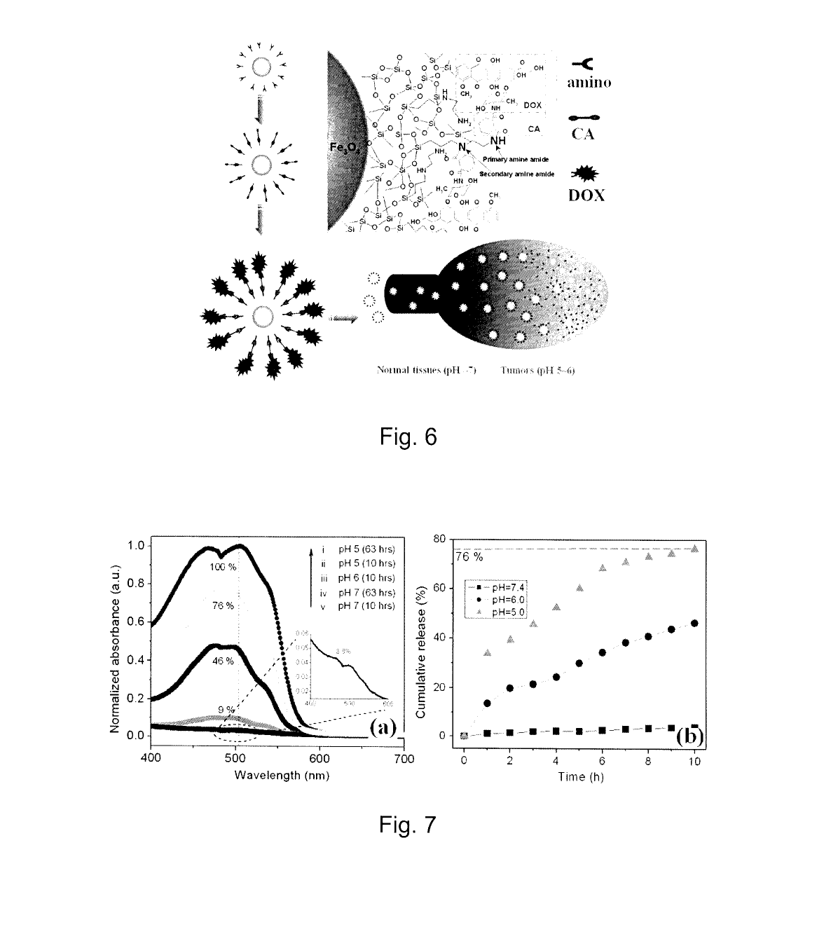

Doxorubicin is one of the most widely used anticancer drugs. However, it is limited by dose-dependent toxic side effects (Crowe 2002). Thus, targeted drug delivery, providing therapeutically effective drug release at the tumor site, exhibits immense potential to resolve this issue and improve the treatment of cancers. The coupling and pH-dependent hydrolysis properties of doxorubicin molecules with primary and secondary amine groups, via 1,2-cyclohexanedicarboxylic anhydride as linkers, have been reported previously (Morris 1978; Xu 2007a). The amides with neighboring carboxylic acid groups are stable at neutral pH, while at a low pH they become negatively charged to regenerate the amine groups and release the free doxorubicin molecules. In line with this we developed a magnetically-guided pH-regulated drug delivery carrier based on Fe₃O₄/silica(porous) nanoparticles. As shown in FIG. 6, the Fe₃O₄/silica(porous) nanoparticles with primary and secondary amine groups were preliminarily functionalized by grafting 1,2-cyclohexanedicarboxylic anhydride molecules. The other sides of 1,2-cyclohexanedicarboxylic anhydride molecules were subsequently coupled to the amine groups of doxorubicin molecules, forming the same amide bonds with neighboring carboxylic acid groups. Under normally physiological conditions (pH about 7), the coupled doxorubicin molecules are quite stable, while can be released by mild acid hydrolysis when they were transported into the region of cancerous tissues (pH 5 to 6) (Xu 2007a; Casasüs 2008; Aznar 2009).

To prepare Fe₃O₄/silica(porous) nanoparticles graft 1,2-cyclohexanedicarboxylic anhydride, the following procedure was used. 2 mg Fe₃O₄/silica(porous) nanoparticles were dissolved in 20 mL DMSO, followed by sonicating for 30 min. Triethylamine (100 μL) was subsequently added and magnetically stirred for 2 h. The grafted nanoparticles were separated by centrifuged at 9000 rpm, and mildly washed by DMSO for three times. The grafted nanoparticles and doxorubicin hydrochloride salt (1 mg) was dissolved in 20 mL DMSO solution, and magnetically stirred for 2 h. In order to remove the free doxorubicin molecules, the doxorubicin-coupled nanoparticles were separated by centrifugation and mildly washed by phosphoric acidic buffer solutions (pH 7.4) three times. The release of doxorubicin from coupled Fe₃O₄/silica(porous) nanoparticles was carried out at 37° C. and in pH 7.4, 6.0 and 5.0 phosphoric acidic buffer solutions, respectively. The separated supernatant solution was monitored by UV-Vis spectrometry.

UV-Vis spectra show the characteristic peaks of doxorubicin molecules at 450 nm to 550 nm as shown in FIG. 7(a), confirming that the coupling and release processes are pH-dependent at 37° C. Concentration of the released doxorubicin was examined by comparing the normalized absorbance intensity of separated supernatant solutions. Based on the intensity at 504 nm, the amount of coupled doxorubicin was about 13.2 mg/100 mg nanoparticles, while the released amount of doxorubicin molecules, at pH 5.0 for 63 hrs, was estimated to be about 9.8 mg/100 mg nanoparticles. The intensity recorded after releasing for 63 h at 37° C. and at pH 5.0 was normalized to be 100% because the peak intensity was almost unchanged for a longer time release. FIG. 7(b) shows the release profiles of doxorubicin molecules at 37° C. as a function of time and pH. It can be seen that a slow release was obtained at pH 7.4, while a drastic increase occurring at pH 6.0 and 5.0. The releasing process, at pH 5.0, initially proceeded relatively fast, and gradually reached equilibrium. Within 6 hrs, 70% of the total release of doxorubicin molecules was attained, with the maximum extent of release being 76% after 10 hrs. At pH 7.4, only about 3.8% of the doxorubicin molecules was released after 10 hrs, far less than the 76% and 46% achieved at pH values of 5.0 and 6.0, respectively. These data reveal that the release of doxorubicin molecules from the Fe₃O₄/silica(porous) nanoparticles exhibit a pH-dependent feature. Since the release rate of doxorubicin molecules at pH 7.4 was significantly lower than at pH 5.0, the majority of the active drug could be released only in cancerous tissues or their intracellular compartments due to a pH decrease in the endosomes and lysosomes. Such controllable release behavior can be used to meet the desirable requirements of drug delivery.

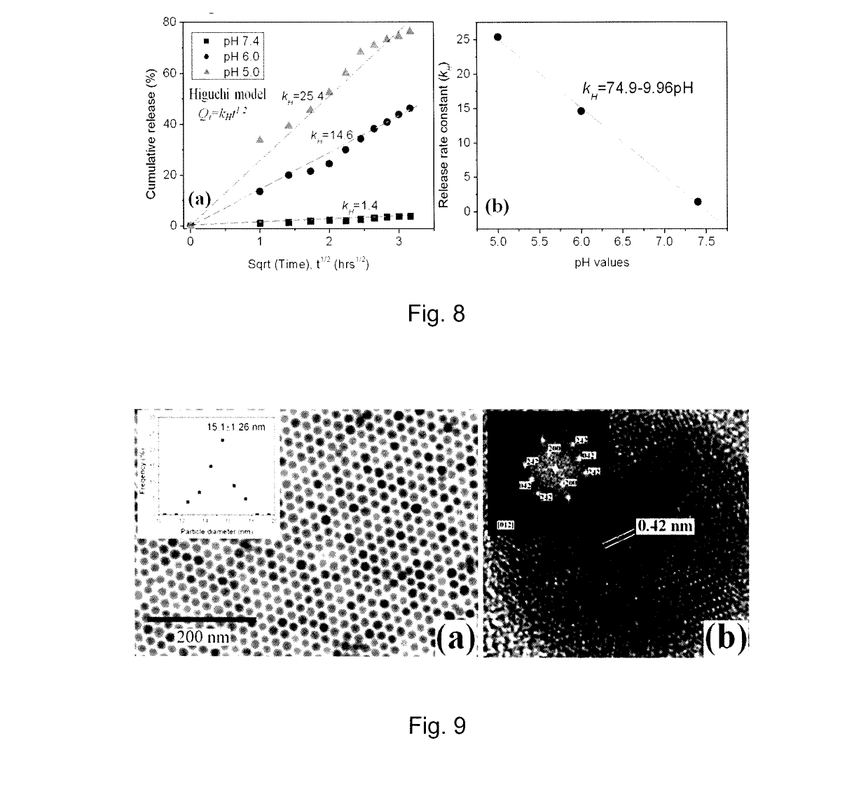

The release of doxorubicin molecules from the pores of the Fe₃O₄/silica(porous) nanoparticles is dominated by a Fickian diffusion kinetic process that can be explained by the Higuchi model, Qₜ=k_{H}t^{1/2}, where Qₜ is the amount of guest release, t is time, k_{H} is the release rate constant (Higuchi 1963; Higuchi 1962; Aznar 2009; Vallet-Regí 2007). The release rate constant (k_{H}) can be further expressed as: k_{H}=2CₒD^{1/2}/π^{1/2}, where Cₒ is the initial concentration of drug in matrix, and D is the diffusion coefficient. The diffusion coefficient (D) is usually a constant when the temperature and the structure of matrix are fixed. In our cases, the changes of release rate constant (k_{H}) at various pH is therefore mainly caused by initial concentration of drug in matrix (Cₒ) that are inversely dependent on pH, as shown in FIG. 8(a). That is to say, the hydrolysis rate of the amides as pH is a vital factor in controlling the release. At pH 7.4, a small amount of amides can be hydrolyzed, resulting in low concentration of free doxorubicin molecules, while at pH 5 and 6, more hydrolyzed amides contribute higher initial concentrations. FIG. 8(b) further shows a linear correlation between the release rate constant (k_{H}) and pH at 37° C. Based on this, one can roughly evaluate the approximate release rate constant (k_{H}) and the release amount (Qₜ) at various time and pH, giving a theoretical direction in practical applications.

### Examples 9-13

### Hollow Nanoporous Magnetic Nanoparticles Functionalized with Secondary Amine

In Examples 9-13, a pH-regulated drug delivery carrier based on superparamagnetic nanoporous core/shell Fe₃O₄/silica hollow nanoparticles (Fe₃O₄/silica(H)) is described in which guest molecules are loaded on nanoporous amino-functionalized silica shells. These nanoparticles, combining large loading ability, hollow architecture, magnetic cores and functional amine groups, exhibit promising potential as drug carriers. To demonstrate this concept, Fe₃O₄/silica(H) nanoparticles functionalized with 1,2-cyclohexanedicarboxylic anhydride as click linkers are effectively coupled to an anticancer drug (doxorubicin) to form amides with neighboring carboxylic acid groups. Importantly, the amide bond was found to be relatively stable at neutral pH 7.4, but can be rapidly hydrolyzed in the range of pH 5.0-6.0. Because normal tissues have a pH of about 7, the majority of doxorubicin can be magnetically delivered and released only in cancerous tissues, which have a pH of about 4 to 6. Due to the porous architecture of silica shells, guest drug molecules not only can be loaded on the surface of the silica shells both outside and inside the nanopores, but can also be loaded into the cavities of the Fe₃O₄/silica(H) nanoparticles. Such multifunctional nanoparticles are very useful in biomedical applications, particularly for magnetically-targeted drug delivery.

### Example 9

### Synthesis of Secondary Amine Functionalized Nanoporous Core/Shell Fe₃O₄/Silica(H) Nanoparticles

Oleic acid-coated Fe₃O₄ (Fe₃O₄/OA) nanoparticles having a mean diameter of about 15 nm were synthesized by thermal decomposition of iron pentacarbonyl as in Example 1 based on a well-known process (Woo 2004). Core/shell Fe₃O₄/silica nanoparticles were synthesized by hydrolyzing TEOS in a water-in-oil microemulsion that contained Fe₃O₄/OA nanoparticles as seeds. Thus, purified Fe₃O₄/OA nanoparticles were first dispersed in cyclohexane at a concentration of 1 mg/mL, and then 0.5 ml of the Fe₃O₄-containing cyclohexane solution was rapidly injected into a mixture of 1.77 g Triton™ X-100, 1.6 ml anhydrous 1-hexanol and 7 ml cyclohexane under a strong vortex for about 1 hour. Subsequently, 0.5 mL of about 6% ammonia solution was added to the above solution and shaken for another 1 hour. Finally, 25 μl TEOS was added and the mixture was allowed to react for 24 h. To the Fe₃O₄/silica nanoparticles so formed, 25 μl of N-(trimethoxysilylpropyl)polyethylenimine (PS076) was added and the reaction continued for another 24 hours under the same conditions. The as-synthesized Fe₃O₄/silica(H) nanoparticles were separated by adding excess ethanol and centrifuging at 9000 rpm for 30 min, which was repeated at least three times. The resultant product was dried under vacuum, or directly dispersed in de-ionized water for characterization.

FIG. 9(a) shows a self-assembly transmission electron microscopy (TEM) image of Fe₃O₄/OA nanoparticles. Almost all the particles are spherical in shape with a uniform size distribution. Based on about 200 particles, it is estimated that the mean diameter is about 15.1 nm with a small deviation of 1.26 nm, and the particle size distribution can be well fitted by a Lorentzian curve, as shown in the inset of FIG. 9(a). FIG. 9(b) shows a high-resolution TEM image of an individual particle. It obviously reveals that the nanoparticle is highly crystalline extending to the outer edges, and the lattice distance is equal to 0.42 nm, corresponding to the (200) plane of Fe₃O₄ phase. Moreover, it can be seen that the fast Fourier transform (FFT) pattern is a symmetrical lattice (inset in FIG. 9b) indicating the single crystalline nature.

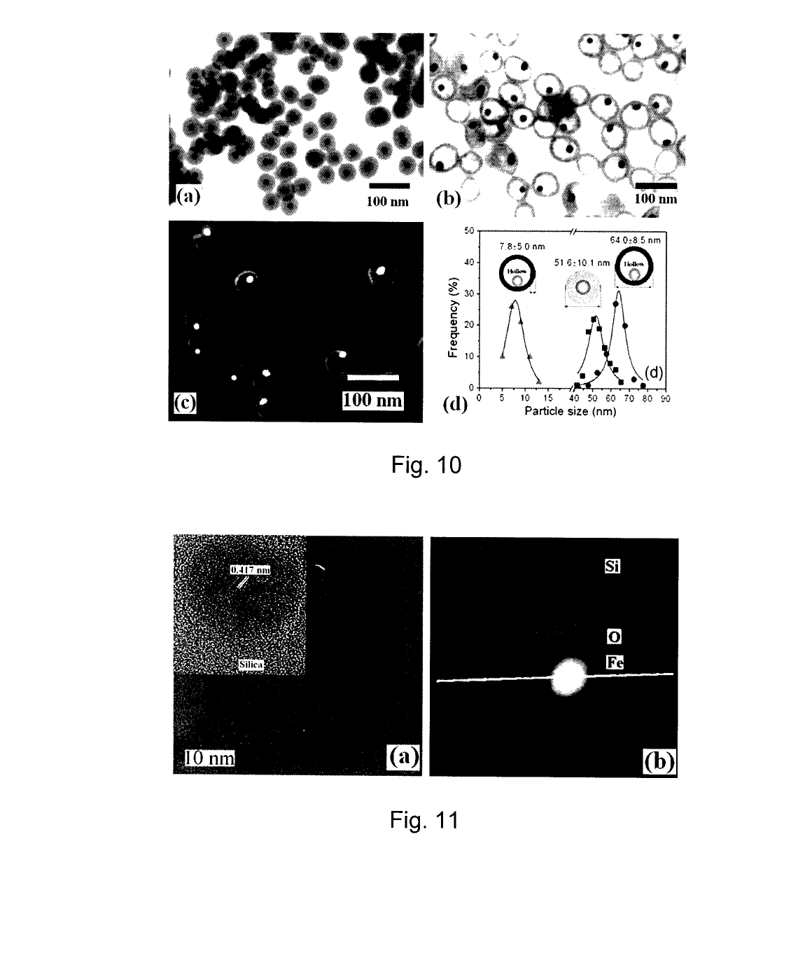

FIG. 10(a) and FIG. 10(b) show TEM images of Fe₃O₄/silica nanoparticles and Fe₃O₄/silica(H) nanoparticles, respectively. As shown in the FIG. 10(a), Fe₃O₄ cores were completely encapsulated in a silica shell with a mean shell thickness of about 18 nm, and exhibited a high uniformity of the core/shell structure and a good mono-dispersersibility in water. Interestingly, hollow nanostructures were obtained after the subsequent amino-functionalized silica coating process by in-situ hydrolyzing PS076 molecules, as shown in FIG. 10(b), which have a more complicated core/shell morphology compared with that in FIG. 10(a). From the TEM images, it is obvious that Fe₃O₄ nanoparticles were not located in the centers of the hollow shells, and tended to stick to the internal walls of shells. Besides the Fe₃O₄/silica(H) nanoparticles, there were some hollow silica shells without Fe₃O₄ cores, which may be a side product of pure silica nanoparticles in the Fe₃O₄/silica product. FIG. 10(c) shows a high angle annular dark field (HAADF) image of Fe₃O₄/silica(H) nanoparticles, providing a clearer contrast for the hollow structure. The size and shape of Fe₃O₄ cores are essentially unchanged. Moreover, a few of the silica shells collapsed, implying that the preliminary silica shells may play a role of sacrificial template in the reaction process. By a combination measurement from bright- and dark-field images, it was estimated that the thickness of the amino-functionalized silica shells and the diameter of the Fe₃O₄/silica(H) nanoparticles were about 7.8 nm and 64.0 nm, respectively, with narrow deviations as shown in FIG. 10(d).

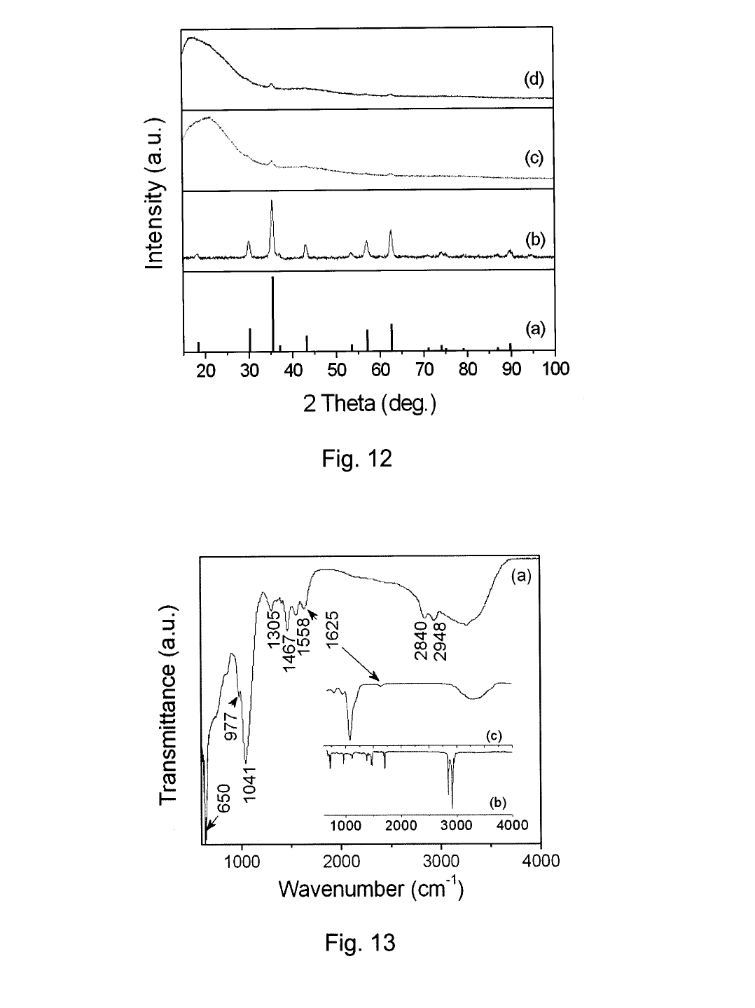

FIG. 11(a) and FIG. 11(b) show magnified TEM images and energy dispersive X-ray spectroscopy (EDS) of an individual Fe₃O₄/silica(H) nanoparticle. Elemental analyses reveal an obvious core/shell feature, in which the core is composed of Fe and O, and the shell is made of Si and O. More interestingly, there is a hollow region between the core and shell. The HRTEM in the inset of FIG. 11(a) indicates that a lattice spacing of 4.17 Å indexed in the core region basically corresponds to the (200) planes of face-centered cubic (FCC) Fe₃O₄, while the silica shells are amorphous. FIG. 12(a)-(d) show XRD patterns of standard Fe₃O₄ powder diffraction card (JCPDS 880315), Fe₃O₄/OA, Fe₃O₄/silica and Fe₃O₄/silica(H) nanoparticles, respectively. All the diffraction peaks are agree well with that of standard Fe₃O₄ powders without appearance of other iron oxides. Chemical analysis result from Guelph Chem. Lab. Ltd. shows that Fe₃O₄/OA nanoparticles contain about 62.5 wt % iron, corresponding to 86.3 wt % Fe₃O₄ and 13.7 wt % OA regardless of other impurities. Both Fe₃O₄/silica and Fe₃O₄/silica(H) nanoparticles comprise an Fe₃O₄ phase and a silica phase, with a characteristic peak of silica appearing at about 20°. Thus, the processes did not cause noticeable changes in the size and structures of Fe₃O₄ nanoparticles, further confirming the direct observations by HRTEM.

Without being held to any particular mechanism, the following is a possible mechanism to explain the formation of silica hollow shells. A typical water-in-oil micro-emulsion system usually comprises oil, water and a surfactant, of which the aqueous phase may contain salt(s) and/or other ingredients. In the present examples, Triton™ X-100 molecules were used as the surfactant that form a monolayer at the water-in-oil interface, with the hydrophobic tails pointing towards the oil phase and the hydrophilic polyethylene oxide heads (PO) in the aqueous phase. With the addition of Fe₃O₄ nanoparticles, the PO heads may be strongly anchored to the surface of the nanoparticles by replacing OA molecules, which brings the Fe₃O₄ nanoparticles into the aqueous reaction phase for subsequent formation of silica shells. It should be noted that in the beginning of the reaction, Triton™ X-100 molecules also play another role in that it limits condensation of TEOS molecules to a solid shell because they are partly retained on the surface of nanoparticles, resulting in a “hybrid” silica shell, i.e. silica debris. Afterward, the ≡Si—OH groups of silica debris make the Fe₃O₄/silica nanoparticles hydrophilic, and disperse them in the aqueous reaction phase. Subsequently, added PS076 molecules with a long chain backbone structure are hydrolyzed and directly react with the silica debris. After removing the Triton™ X-100 molecules and silica debris by ethanol washing, the cavities of Fe₃O₄/silica(H) form and are finally retained due to steric hindrance by backbones of hydrolyzed PS076.

### Example 10

### Surface Characterization of Nanoporous Fe₃O₄/Silica(H) Nanoparticles

FIG. 13(a), FIG. 13(b) and FIG. 13(c) show Fourier transmission infrared (FTIR) spectra of the Fe₃O₄/silica(H), Fe₃O₄/OA, and Fe₃O₄/silica nanoparticles. The FTIR spectrum of Fe₃O₄/silica(H) nanoparticles indicates a characteristic peak for formation of vₐₛ ≡Si—O—Si≡ bonds at 1041 cm⁻¹, and a stretching vibration of v ≡Si—OH bonds at about 977 cm⁻¹, confirming the incomplete condensation of PS076 molecules. Moreover, characteristic peaks of adsorbed water and ethanol were found in Fe₃O₄/silica(H) and Fe₃O₄/silica nanoparticles, such as δ HOH at 1625 cm⁻¹, vₐₛ ≡CH at 1467 cm⁻¹, and v —OH at 3000-3500 cm⁻¹ (Jitianu 2003). Comparing the spectra of Fe₃O₄/OA nanoparticles to Fe₃O₄/silica nanoparticles, it is apparent that oleic acid molecules were completely removed after silica shell formation since there is an absence of all the characteristic OA peaks in the FTIR spectrum of Fe₃O₄/silica nanoparticles. It should be emphasized that the characteristic peaks of secondary amine groups appear at 650 cm⁻¹ (V_{out-of-plane} NH), 1305 cm⁻¹ (vₐₛ CN), 1558 cm⁻¹ (vᵢₙ₋ₚₗₐₙₑ NH), 2948 cm⁻¹ ((vₐₛ ═CH₂) and 2840 cm⁻¹ ((vₐₛ —CH in —CH₂—NH—CH₂—), indicating that the hollow silica shells are from the hydrolysis of PS076 molecules (Simons 1978).

### Example 11

### Magnetic Properties of Nanoporous Fe₃O₄/Silica(H) Nanoparticles

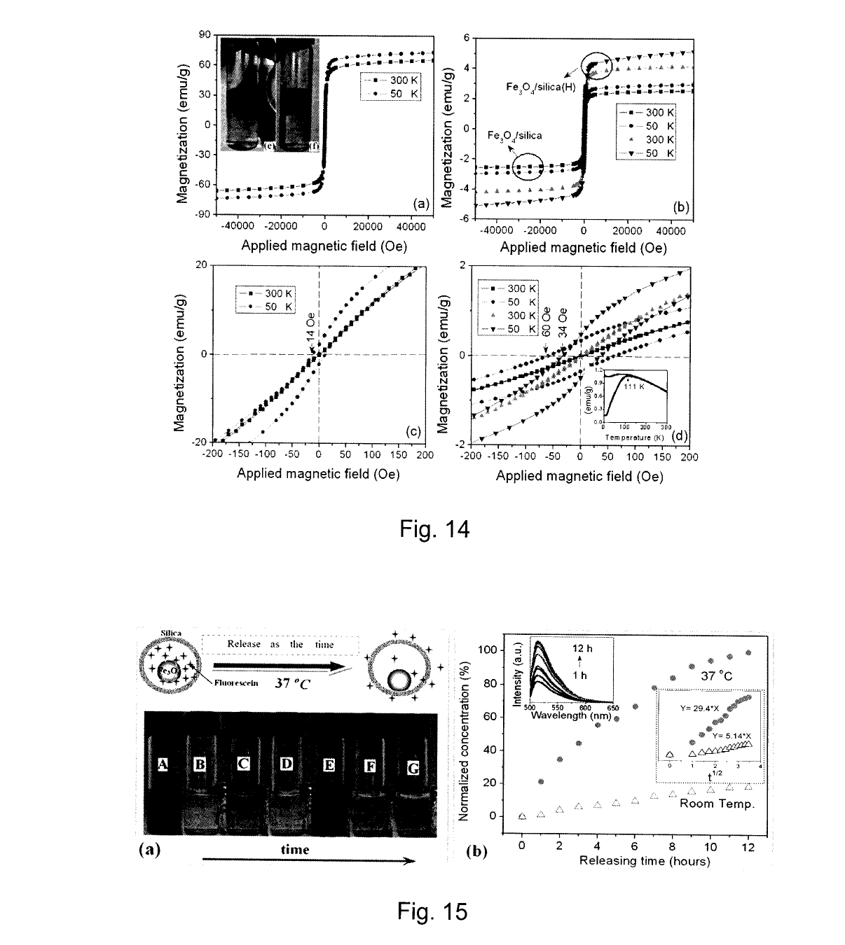

FIG. 14 shows hysteresis loops and the corresponding magnification at origin of Fe₃O₄/OA, Fe₃O₄/silica and Fe₃O₄/silica(H) nanoparticles at measurement temperatures of 300 K and 50 K. The Fe₃O₄/OA nanoparticles in FIG. 14(a) and FIG. 14(c) present ferromagnetic properties with a saturated magnetization of about 73 emu/g and a small magnetic coercivity of 14 Oe at 50 K. The mean particle size of Fe₃O₄/OA nanoparticles is about 15 nm, which is far below the critical size (about 25 nm) for the magnetic transition from superparamagnetic to ferromagnetic (Park 2004; Sun 2004). The coercivity and remnant magnetization were observed to be zero at 300 K, indicating that the Fe₃O₄/OA nanoparticles are superparamagnetic. Fe₃O₄/OA nanoparticles are hydrophobic and can be easily dispersed in hexane without any evidence of aggregation. In a high concentration, they can form a magnetic fluid and exhibit a rapid magnetic response. The dispersed nanoparticles can also be easily separated from hexane by adding ethanol when the solution sample is close to a permanent magnet. However, the saturated magnetizations of Fe₃O₄/silica and Fe₃O₄/silica(H) nanoparticles were reduced down to about 3 emu/g and about 5 emu/g, respectively, due to the non-magnetic silica compositions, as shown in FIG. 14(b). It can be noted that the saturated magnetization of Fe₃O₄/silica(H) nanoparticles is much bigger than that of Fe₃O₄/silica nanoparticles, implying that removed silica debris was successfully separated from the product by washing and magnetic separations. Increased saturated magnetization offers a more significant feature for magnetic manipulation in various applications. Quite interestingly, the coercivity of Fe₃O₄/silica and Fe₃O₄/silica(H) nanoparticles increased to 60 Oe and 34 Oe at 50 K, bigger than that of the Fe₃O₄/OA nanoparticles, although they still retained superparamagnetic features. The enhancement of coercivity is mainly ascribed to a weakened interaction between magnetic Fe₃O₄ cores depending on the topology of the shells, which contributes to a mono-dispersibility in solution. The inset in FIG. 14(d) shows the temperature-dependent zero-field-cooling (ZFC) and field-cooling (FC) magnetization curve of Fe₃O₄/silica(H) nanoparticles in an applied magnetic field of 50 Oe, exhibiting a broader maximum of about 111 K.

### Example 12

### Validation of Temperature-Dependent Release from Fe₃O₄/Silica(H) Nanoparticles

The hollow architecture of Fe₃O₄/silica(H) nanoparticles makes them particularly suitable as drug carriers for applications in magnetically-targeted delivery and release. Considering the non-interaction of fluorescein molecules with nanoparticles, it was selected to validate temperature-dependent release. Dependence of release on time and temperature was examined by re-dispersing fluorescein-doped Fe₃O₄/silica(H) nanoparticles in water, and comparing the normalized intensity of emission at 515 nm after magnetic separation. The intensity recorded after releasing for 12 hours at 37° C. was normalized to be 100%. FIG. 15(a) shows a schematic diagram for the releasing process, and optical photographs at various releasing stages at 37° C. followed by magnetic separation, confirming an effective release based on color changes of solutions. FIG. 15(b) shows the release percentage of fluorescein molecules at room temperature and 37° C. as a function of time. It can be seen that a slow release was obtained at room temperature, while a drastic increase was observed at 37° C. The releasing process at 37° C. initially proceeded relatively fast, and gradually reached equilibrium after 8 hours. Assuming that the release is dominated by a simple diffusion kinetic process from the cavities of the Fe₃O₄/silica(H) nanoparticles, the release can be explained by the Higuchi model, Qₜ=k_{H}t^{1/2} (Higuchi 1962; Higuchi 1963), where Qₜ is the amount of guest release, t is time, k_{H} is the release rate constant. Porous silica systems for non-controlled release of drugs have been satisfactorily explained by the Higuchi mode (Aznar 2009; Vallet-Regí 2007). As expected, the released amount at room temperature and 37° C. can be well fitted linearly via the square root of time with a release rate constants (k_{H}) of 5.14 and 29.4 (see the lower inset of FIG. 15(b)). The release of fluorescein molecules, in accordance to the Higuchi model, clearly shows that guest drug molecules can be loaded into the cavities of Fe₃O₄/silica(H) nanoparticles, which may be ascribed to the porous architectures of silica shells.

### Example 13

### pH-Regulated Doxorubicin Release from Nanoporous Fe₃O₄/Silica(H) Nanoparticles

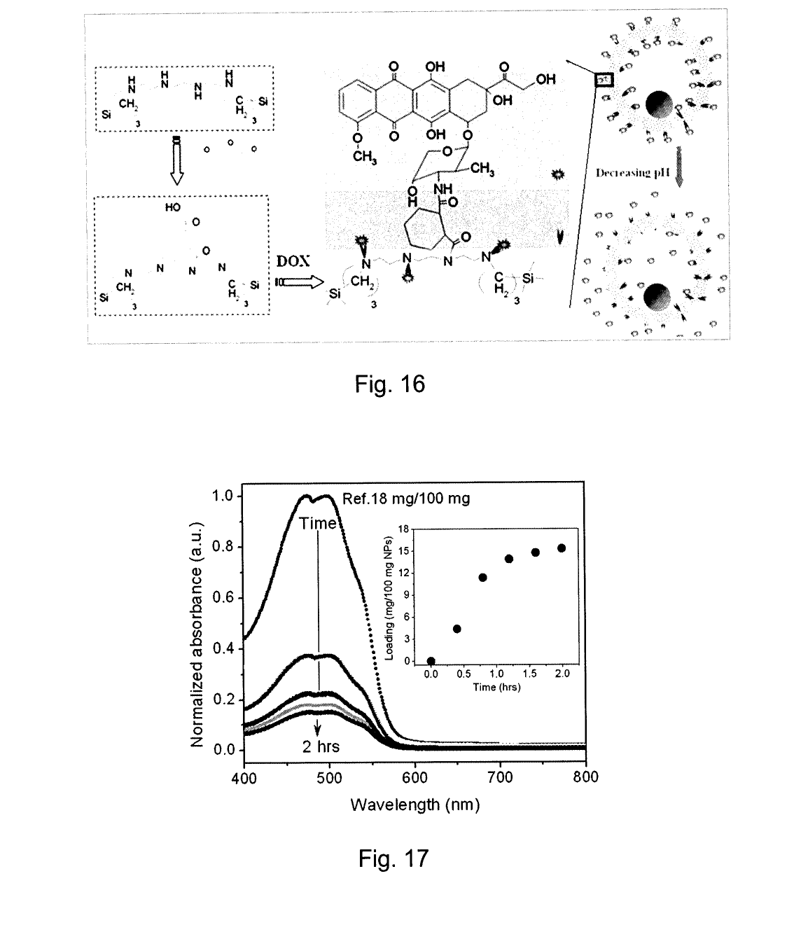

As one of the most widely used anticancer drugs, doxorubicin has exhibited a broad spectrum of activity against solid tumors. The therapy, however, is limited by dose-dependent toxic side effects which can potentially lead to heart failure due to the cardiotoxicity (Crowe 2002). Targeted drug delivery, providing therapeutically effective drug release at the tumor site, is an effective solution and improves the treatment of cancers. The conjugation scheme of doxorubicin with secondary amides, via 1,2-cyclohexanedicarboxylic anhydride as linkers, is as shown in FIG. 16. The nanoporous silica shells of the Fe₃O₄/silica(H) nanoparticles are first treated with 1,2-cyclohexanedicarboxylic anhydride to provide terminal carboxylic groups on the shell. The carboxylic groups then react with amine groups of doxorubicin molecules to form amide linkages, which effectively serve as pH-triggered switches due to the effect of neighboring carboxylic acid groups. The amide linkages are chemically stable at neutral pH, while at a low pH they become negatively charged to regenerate the amine groups (Xu 2007a; Morris 1978). Therefore, doxorubicin molecules can be very easily released by decreasing the pH.

To prepare Fe₃O₄/silica(H) nanoparticles graft 1,2-cyclohexanedicarboxylic anhydride, the following procedure was used. 2 mg Fe₃O₄/silica(H) nanoparticles were dissolved in 20 mL DMSO, followed by sonicating for 30 min. Triethylamine (100 μL) was subsequently added and magnetically stirred for 2 hours. The grafted nanoparticles were separated by centrifuged at 9000 rpm, and mildly washed with DMSO for three times. The grafted nanoparticles and doxorubicin hydrochloride salt (1 mg) were dissolved in 20 mL DMSO solution, and magnetically stirred for 2 hours. In order to remove free doxorubicin molecules, the doxorubicin-coupled nanoparticles were separated by centrifugation and mildly washed three times with pH 7.4 phosphoric acidic buffer solutions. The release of doxorubicin from coupled Fe₃O₄/silica(H) nanoparticles was carried out at 37° C. and room temperature, and at pH 7.4, 6.0 and 5.0 in phosphoric acidic buffer solutions, respectively. The separated supernatant solution was monitored by UV-V is spectroscopy.

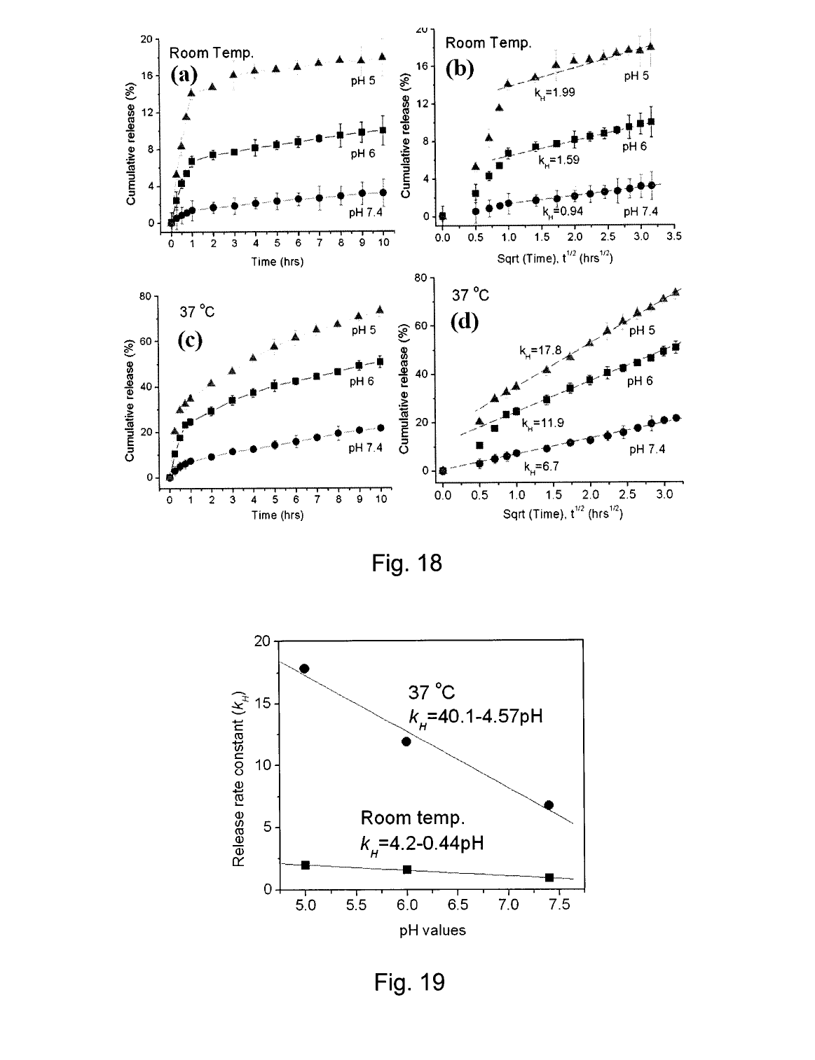

UV-Vis absorbance spectra of separated supernatant solution of Fe₃O₄/silica(H) nanoparticles were measured before and after loading doxorubicin molecules, as shown in FIG. 17. The loading mass of doxorubicin molecules was calculated to be 15.3 mg/100 mg Fe₃O₄/silica(H) nanoparticles for a loading time of 2 hrs, which is normalized to be 100%. To estimate the payload of doxorubicin molecules, release behaviors dependent on pH and temperature were studied as shown in FIG. 18. At room temperature, the released content of doxorubicin molecules at pH 7.4 after 10 hrs is quite low, only 3.2%, indicating that the amides are stable enough to trap doxorubicin molecules. Released content increases almost immediately to 6.7% and 14.0% within 1 hour when the pH is 6 and 5. To further elucidate the release mechanism, the release profile was plotted based on the Higuchi model, as shown in FIG. 18(b). The release rate constants (k_{H}) can be linearized for the initial 1 hour, or from 1 to 10 hrs. The later release rate constant is about 1.99 at pH 5, about 2 times larger than that at pH 7.4 (0.94). In order to better simulate physiological conditions, release behaviors were studied at 37° C. in PBS buffers. From FIG. 18(c) and FIG. 18(d), it can be seen that there is a rapid release of doxorubicin molecules at pH 5 and 6 within 1 hour, similar to room temperature. The release content of 1 hour at pH 5, 6 and 7.4 were estimated to be 34.7%, 24.5% and 7.2%, respectively. The rapid release in the initial stage is mainly a consequence of the doxorubicin molecules coupled on the surface of the nanoparticles, which can more easily diffuse into the buffer after hydrolysis of the amide linkage compared with the doxorubicin molecules loaded into the hollow cavities of the nanoparticles. In contrast, fluorescein molecules physically adsorbed on the surface of Fe₃O₄/silica(H) nanoparticles as in Example 12 were effectively removed by washing, so that similar phenomena did not occur. From 1 to 10 hrs, the release content stably increases and reaches a highest value of 73.2% at pH 5. Moreover, these data as a function of the square root of time (1-10 hrs) as shown in FIG. 18(d) are in good agreement with the linear relationship of the Higuchi model with the corresponding release rate constants of 17.8, 11.9 and 6.7 at pH 5, 6 and 7.4, respectively.

In the Higuchi model, the release rate constant (k_{H}) can be expressed as: k_{H}=2CₒD^{1/2}/π^{1/2}, where Cₒ is the initial concentration of drug in matrix and D is the diffusion coefficient. The diffusion coefficient (D) is related to temperature and the structure of the matrix, which are fixed in the present cases. It is reasonable to assume that the change of release rate constant (k_{H}) at various pH is mainly caused by initial concentration of drug in matrix (Cₒ). It should be noted that the release rate constants of doxorubicin molecules are inversely dependent on pH, suggesting that the pH plays a critical role in controlling the release. The coupling and hydrolysis of the amide linkages are pH-dependent. At pH 7.4, a small amount of doxorubicin molecules can be released because most of them are bound by amide linkages, resulting in low concentration of free doxorubicin molecules, i.e. an “effective initial concentration”, while at pH 5 and 6, more hydrolyzed amides contribute to higher effective initial concentrations. FIG. 19 shows the correlation between the release rate constant (k_{H}) and pH at room temperature and 37° C.

### Examples 14-16

### Core/Shell Fe₃O₄/Silica(SH) Magnetic Nanoparticles and Functionalization to Target Delivery of Nitric Oxide (NO) Molecules

In Examples 14-16, a new nitric oxide (NO) molecule delivery carrier was developed based on thiol-functionalized Fe₃O₄/silica NPs (denoted below as Fe₃O₄/silica(SH)), in which NO molecules are grafted to the —SH groups of the silica shells. These nanoparticles, combining large loading ability, magnetic cores and functional —SH groups, exhibit promising potential as NO carriers. To demonstrate this concept, the conjugation of NO molecules by transforming —SH groups to —SNO groups of Fe₃O₄/silica(SH) nanoparticles are developed by reacting the —SH functional groups with t-butyl nitrite or NaNO₂ to form —SNO groups. The —SNO groups of Fe₃O₄/silica(SNO) nanoparticles were found to be relatively stable at low temperature less than 4° C. and light-shielding condition, but can be stably released by extra-stimuli such as ultrathin gold nanoparticles and/or temperature (even at room temperature). Such multifunctional nanoparticles are very useful in biomedical applications, particularly for magnetically-targeted drug delivery.

### Example 14

### Synthesis of Core/Shell Fe₃O₄/Silica(SH) Nanoparticles

Thiol-functionalized Fe₃O₄/silica NPs (denoted as Fe₃O₄/silica(SH)) may be synthesized with improved density of thiol groups. Based on the protocol in Examples 1 and 2, —OH group functionalized silica-coated NPs (Fe₃O₄/silica(—OH)) can be obtained. Fe₃O₄/silica(SH) NPs can then be obtained by in situ condensation in and on the surface of the Fe₃O₄/silica(—OH) NPs by using 3-mercaptopropyltrimethoxysiliane or other silanes with thiol groups. The detailed protocol contains a two-step procedure involving hydrolyzing TEOS and 3-mercaptopropyltrimethoxysilane molecules. The first step comprised the synthesis of Fe₃O₄/silica(1) nanoparticles of Example 2 by hydrolyzing TEOS. After forming silica shells, 1-5 μl of 3-mercaptopropyltrimethoxysilane were injected into the reaction mixture for another 24 h. The resultant product was denoted as Fe₃O₄/silica(—SH) nanoparticles. The products were centrifuged at 9000 rpm and washed with anhydrous ethanol three times, and finally dispersed in de-ionized water for use.

As another alternative, the silica shell of Fe₃O₄/silica(1) NPs of Example 2 are functionalized by the post-modification procedure. For example, 20 mg Fe₃O₄/silica(1) NPs and various quantity (1-5 μl) of 3-mercaptopropyltrimethoxysilane are dispersed in 10 ethanol by ultrasonication, and heating up to 60° C. for 24 hrs. The product (Fe₃O₄/silica(SH)) was centrifuged at 9000 rpm and washed with anhydrous ethanol three times, and finally dispersed in de-ionized water for use.

### Example 15

### Nitric Oxide (NO) Molecule Conjugation of Core/Shell Fe₃O₄/Silica(SH) Nanoparticles

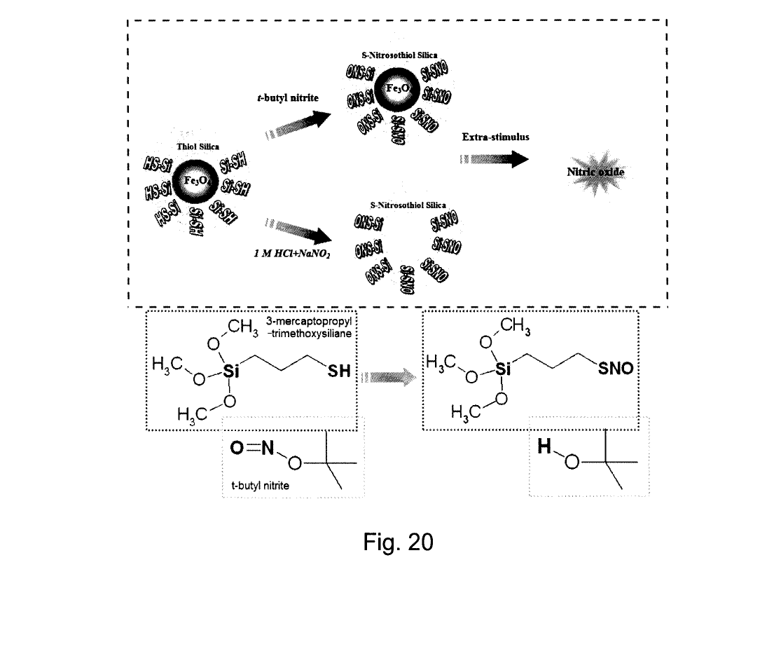

To conjugate the NO molecules on the surface of Fe₃O₄/silica(SH) NPs one of two protocols may be used. As shown in FIG. 20, the conjugation of NO molecules by transforming —SH groups to —SNO groups of Fe₃O₄/silica(SH) NPs can occur by reacting the —SH functional groups with t-butyl nitrite (protocol 1) or 1 M HCl+NaNO₂(protocol 2) to form —SNO groups. The detailed procedures are: 20 mg Fe₃O₄/silica(—SH) NPs and excess with t-butyl nitrite (protocol 1) or NaNO₂ (protocol 2) are dispersed in 10 ml de-ionized water or 1 M HCl solution at 0° C., and stirred for 24 hrs. The products with —SNO groups were centrifuged at 9000 rpm and washed with anhydrous ethanol three times, dispersed in de-ionized water or dried, and finally stored at low temperature under light-shielded conditions.

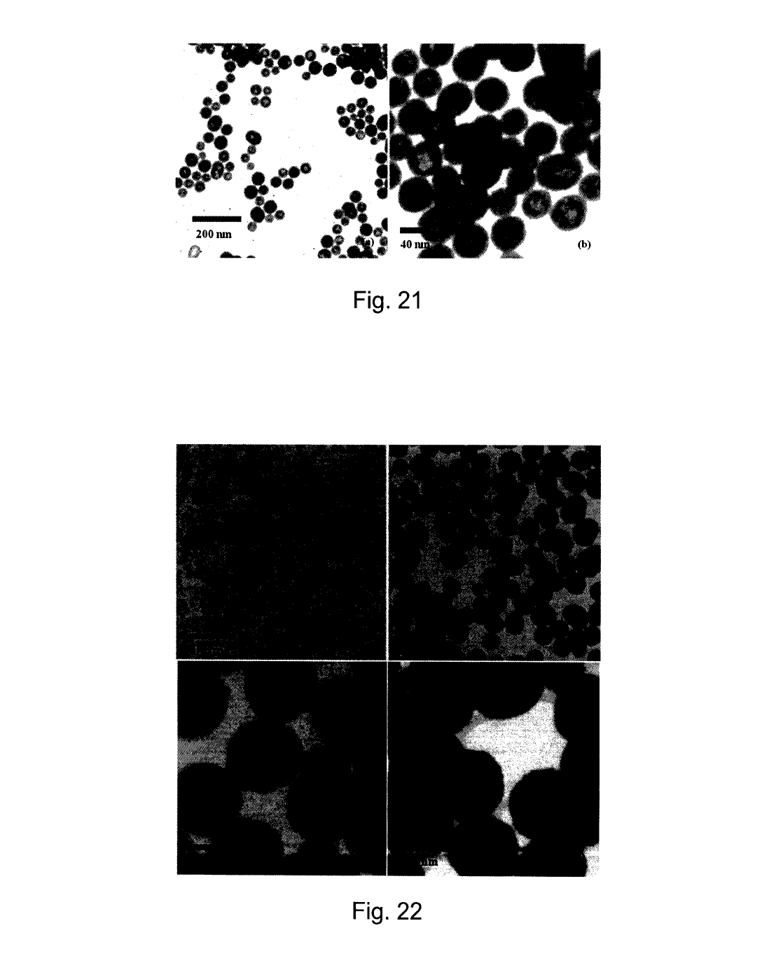

However, when forming the —SNO groups in 1 M HCl+NaNO₂ solution, the Fe₃O₄ cores are dissolved by HCl leaving hollow silica (—SNO) shells as shown in FIG. 21. The Fe₃O₄/silica(SNO) NPs functionalized by t-butyl nitrite (protocol 1) retain complete core/shell structures with the Fe₃O₄ cores, as shown in FIG. 22.

### Example 16

### Determination and Regulated Release of Nitric Oxide Molecules from Fe₃O₄/Silica(SNO) Nanoparticles

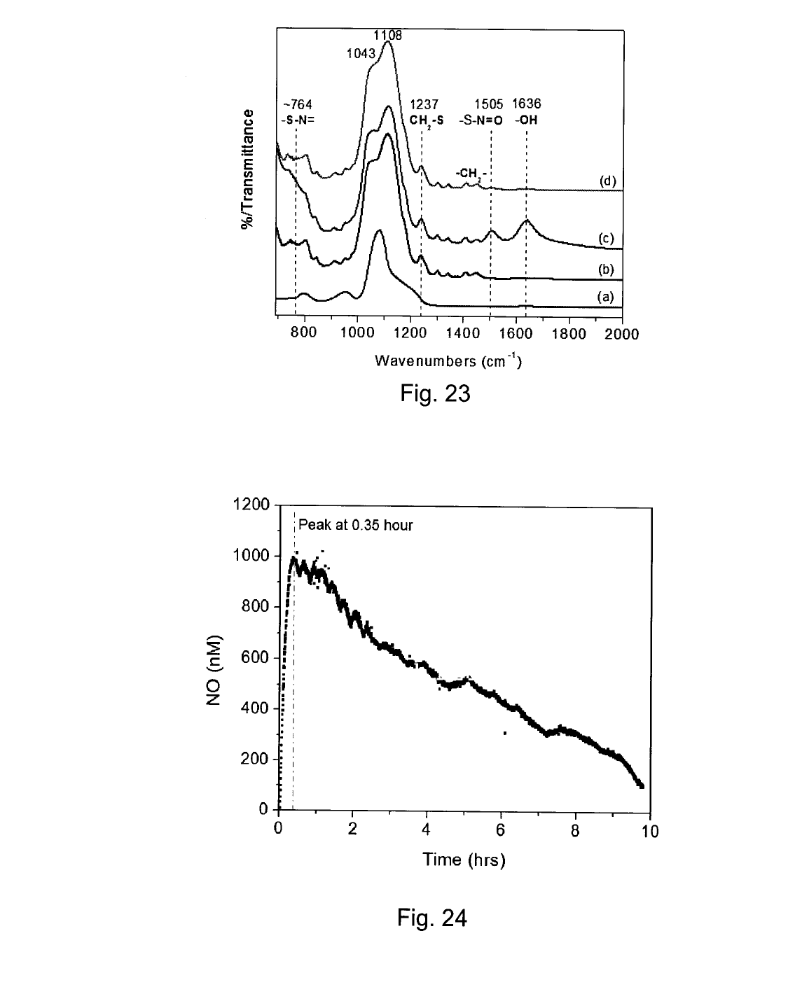

Coupling and release of NO may be triggered by temperature, metal ions and ultrathin Au NPs by FTIR (FIG. 23). The formation of the —S—NO groups on the surface of Fe₃O₄/silica NPs is evidenced by the presence of the characteristic IR peaks of —S—N═, —CH₂—S— and —N═O at 764, 1237 and 1505 cm⁻¹, respectively. After exposure at room temperature for one day these —S—NO characteristic peaks disappear in the FTIR indicating the release of the NO at room temperature after 24 h. The release is much faster when the temperature is increased.

To trigger the release of the NO molecules from Fe₃O₄/silica(—SNO), three routes have been studied in detail. First, temperature-dependent release kinetics of the nanoparticles at low temperature (4° C.), room temperature and normal body temperature (37° C.) was used to evaluate storage and release rates in real practical applications. Second, metal ion triggered release was studied. The —SNO groups may be susceptible to the presence of trace metal ions that are present in body fluids, thus it is quite important to evaluate the effect of NO release by some metal ions such as copper ions. Finally, ultrasmall gold NPs (about 1.5 nm) can be used to control the release of NO, even at 4° C. at which the —SNO groups are relatively stable. Results indicate that the —SNO groups are quickly immobilized on ultrasmall gold nanoparticles by Au—S interactions, as shown in FIG. 22(d). The NPs before and after the release exhibit various colors, providing further evidence.

Detection of NO release of the nanoparticles was carried out by amperometric analysis using the Nitric Oxide Detector (World Precision Instruments). The ISO-NOP sensor (World Precision Instruments Ltd.) was calibrated to be the detection sensitivity of 0.967 pA/nM. Here, 1.5 mg nanoparticles were dispersed into 1 ml PBS 7.2 buffer, forming a stable suspension, and then rapidly injected into 19 ml PBS 7.2 buffer at which moment the ISO-NOP sensor had reached a low and stable current level. The NO probe was immersed about 2 cm into the suspension with magnetic stirring of 600 rpm, and the measurement temperature was fixed at 25° C. As shown in FIG. 24, amperometric analysis revealed immediate NO release after the addition of nanoparticles. It is evident that the rate of NO release is relatively stable with an initial peak of 980 nM at 0.35 hrs, and then the rate gradually decreases with time up to 10 hrs.

### REFERENCES

The contents of the entirety of each of which are incorporated by this reference.
Allia P, Coisson M, Tiberto P, Vinai F, Knobel M, Novak M A, Nunes W C. (2001) Phys. Rev. B. 64, 144420.
Arruebo M, Fernández-Pacheco R, Velasco B, Marquina C, Arbiol J, frusta S, Ibarra M R, Santamaría J. (2007) Adv. Fund. Mater. 17, 1473.
Aznar E, Marcos M D, Martínez-Máńez R, Sancenón F, Soto J, Amorós P, Guillem C. (2009) J. Am. Chem. Soc. 131, 6833.
Back D M, Francombe M H, Vossen J L, Eds. (1991) Physics of Thin Film. (Academic Press, San Diego, Calif.).
Bertoluzza A, Fagnano C, Morrelli M A. (1982) J. Non-Crys. Sol. 48, 117.
Boyer C, Whittaker M R, Bulmus V, Liu J, Davis T P. (2010) NPG Asia Mater. 2(1), 23-30.
Casasüs R, Climent E, Marcos M D, Martínez-Máńez R, Sancenón F, Soto J, Amorós P, Cano J, Ruiz E. (2008) J. Am. Chem. Soc. 130, 1903.
Chattopadhyay P, Gupta R B. (2002) Ind. Eng. Chem. Res. 41, 6049.
Chukin G D, Malevich V I. (1977) J. Appl. Spectroscopy. 26, 223.
Climent E, Bernardos A, Martínez-Máńez R, Maquieira A, Marcos M D, Pastor-Navarro N, Puchades R, Sancenón F, Soto J, Amorós P. (2009) J. Am. Chem. Soc. 131, 14075.
Coluccla F B, Ghlottl G, Morterra C, Zecchina A. (1978) J. Phys. Chem. 82, 1298.
Crowe D L. (2002) Recent Res. Dev. Cancer. 4, 65.
Culler S R, Ishida H, Koenig J L. (1984) Appl. Spectroscopy. 38, 1.
Cullity B D, Ed. (1972) Introduction to Magnetic Materials. (Addison-Wesley, Mass.).
Dormann J L, Fiorani D, Tronc E. (1997) Adv. Chem. Phys. 98, 283.
Duff D G, Baiker A, Edwards P P. (1993a) Langmuir. 9, 2301.
Duff D G, Baiker A, Gameson I, Edwards P P. (1993b) Langmuir. 9, 2310.
Fang C, Bhattarai N, Sun C, Zhang M Q. (2009) Small. 5, 1637.
Feng X, Fryxell G E, Wang L-Q, Kim A Y, Liu J, Kemner K M. (1997) Science. 276, 923.
Fernández-Pacheco R, Arruebo M, Marquina C, Ibarra R, Arbiol J, Santamaría J. (2006) Nanotechnology. 17, 1188.
Fu A, Wang S X, Sam Gambhir S. (2010) United States Patent Publication 2010-0008862 published Jan. 14, 2010.
Gore M, Ed. (2000) Spectrophotometry and Spectrofluorimetry: A Practical Approach. (Oxford University Press, Incorporated).
Guerrero-Martínez A, Pérez-Juste J, Liz-Marzán L M. (2009) Adv. Mater. DOI: 10.1002/adma.200901263.
Gupta A K, Gupta M. (2005) Biomaterials. 26, 3995.
Hetrick E M, Shin J H, Stasko N A, Johnson C B, Wespe D A, Holmuhamedov E, Schoenfisch M H. (2008) ACS Nano. 2, 235.
Hetrick E M, Shin J H, Paul H S, Schoenfisch M H. (2009) Biomaterials. 30, 2782.
Higuchi W I. (1962) J. Pharm. Sci. 51, 802.
Higuchi T J. (1963) J. Pharm. Sci. 52, 1145.
Jitianu A, Britchi A, Deleanu C, Badescu V, Zaharescu M. (2003) J. Non-Crys. Solids. 319, 263.
Kang K, Choi J, Nam J H, Lee S C, Kim K J, Lee S W, Chang J H. (2009) J. Phys. Chem. B. 113, 536.
Ma D L, Guan J W, Normandin F, Dénommée S, Enright G, Veres T, Simard B. (2006) Chem. Mater. 18, 1920.
Mal N K, Fujiwara M, Tanaka Y. (2003) Nature. 421, 350.
Morris J J, Page M I. (1978) J. Chem. Soc. Chem. Commun. 591.
Nguyen T D, Leung K C F, Liong M, Liu Y, Fraser Stoddart J, Zink J I. (2007) Adv. Funct. Mater. 17, 2101.
Nguyen T Q, Wu J J, Doan V, Schwartz B J, Tolbert S H. (2000) Science. 288, 652.
Niu D C, Li Y S, Ma Z, Diao H, Gu J L, Chen H R, Zhao W R, Ruan M L, Zhang Y L, Shi J L. (2010) Adv. Funct. Mater. 20, DOI: 10.1002/adfm.200901493.
Qian H S, Guo H C, Ho P C, Mahendran R, Zhang Y. (2009) Small. 5, 2285.
Park J, An K, Hwang Y, Park J G, Noh H J, Kim J Y, Park J H, Hwang N M, Hyeon T. (2004) Nature Mater. 3, 891.
Patel K, Angelos S, Dichtel W R, Coskun A, Yang Y W, Zink J I, Stoddart J F. (2008) J. Am. Chem. Soc. 130, 2382.
Rickwood D, Ed. (1984) Centrifugation: A Practical Approach. (Oxford University Press).
Santra S, Tapec R, Theodoropoulou N, Dobson J, Hebard A, Tan W. (2001) Langmuir. 17, 2900.
Sappey R, Vincent E, Hadacek N, Chaput F, Boilot J P, Zins D. (1997) Phys. Rev. B. 56, 14551.
Shen Y, Xu P. (2008) International Patent Publication W O 2008-005479 published Jan. 10, 2008.
Shin J H, Metzger S K, Schoenfisch M H. (2007) J. Am. Chem. Soc. 129, 4612.
Simons W W., Ed. (1978) The handbook of infrared spectra. (Philadelphia: Sadtler Research Laboratories Press).
Slowing I I, Trewyn B G, Giri S, Lin V S Y. (2007) Adv. Funct. Mater. 17, 1225.
Stamopoulos D, Manios E, Gogola V, Benaki D, Bouziotis P, Niarchos D, Pissas M. (2008) Nanotechnology. 19, 505101.
Sun S H, Zeng H, Robinson D B, Raoux S, Rice P M, Wang S X, Li G X. (2004) J. Am. Chem. Soc. 126, 273.
Tan W, Santra S, Zhang P, Tapec R, Dobson J. (2003) U.S. Pat. No. 6,548,264 issued Apr. 15, 2003.
Tan W, Shouguang J, Zhao X, Dytioco R T, Drake T J, Hilliard L R. (2004) United States Patent Publication 2004-0067503 published Apr. 8, 2004.
Thornton P D, Heise A. (2010) J. Am. Chem. Soc. 132, 2024.
Torney F, Trewyn B G, Lin V S Y, Wang K. (2007) Nature Nanotech. 2, 295.
Tsunekawa S, Ishikawa K, Li Z Q, Kawazoe Y, Kasuya A. (2000) Phys. Rev. Lett. 85, 3440.
Udenfriend S, Stein S, Bohlen P, Dairman W, Leimgruber W, Weigele M. (1972) Science. 178, 871.
Vadalaa M L, Zalichb M A, Fulksa D B, Pierreb T G S, Daileyc J P, Rifflea J S. (2005) J. Magn. Magn. Mater. 293, 162.
Vallet-Regí M, Balas F, Arcos D. (2007) Angew. Chem., Int. Ed. 46, 7548.
Vargas J M, Nunes W C, Socolovsky L M, Knobel M, Zanchet D. (2005) Phys. Rev. B. 72, 184428.
Wang Y-X, Leung C-F, Qin L. (2009) United States Patent Publication 2009-0297615 published Dec. 3, 2009.
Warren B E, Ed. (1969) X-ray Diffraction. (Addison-Wesley Press, London).
Westcott S L, Oldenburg S J, Lee T R, Halas N J. (1988) Langmuir. 14, 5396.
Woo K, Hong J, Choi S, Lee H W, Ahn J P, Kim C S, Lee S W. (2004) Chem. Mater. 16, 2814.
Xu P S, Van Kirk E A, Zhan Y H, Murdoch W J, Radosz M, Shen Y Q. (2007a) Angew. Chem. Int. Ed. 46, 4999.
Xu Z C, Hou Y L, Sun S H. (2007b) J. Am. Chem. Soc. 129, 8698.
Yi D K, Selvan S T, Lee S S. (2005) J. Am. Chem. Soc. 127, 4990.
Ying J Y, Jana N R, Zheng Y. (2008) United States Patent Publication 2008-0045736 published Feb. 21, 2008.
Yoon T J, Kim J S, Kim B G, Yu K N, Cho M H, Lee J K. (2005) Angew. Chem. Int. Ed. 44, 1068.
Zhang X F, Dong X L, Huang H, Wang D K, Lv B, Lei J P. (2007a) Nanotechnology. 18, 275701.
Zhang X F, Dong X L, Huang H, Lv B, Zhu X G, Lei J P, Ma S, Liu W, Zhang Z D. (2007b) Acta Materialia. 55, 3727.
Zhang M, Cushing B L, O'Connor C J. (2008) Nanotechnology. 19, 085601.
Zhao Y N, Trewyn B G, Slowing I I, Lin V S Y. (2009a) J. Am. Chem. Soc. 131, 8398. Zhao N, Gao M Y. (2009b) Adv. Mater. 21, 184.
Zhou W, Thomas J M, Shephard D S, Johnson B F G, Ozkaya D, Maschmeyer T, Bell R G, Ge Q. (1998) Science. 280, 705.

Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A magnetic nanoparticle comprising: one or more cores comprising a superparamagnetic nanoparticle; and, a nanoporous silica shell surrounding the one or more cores, the shell having nanopores that extend to the surface of the magnetic nanoparticle, the shell functionalized with amine groups both inside and outside the nanopores.

2. The magnetic nanoparticle according to claim 1, wherein the superparamagnetic nanoparticle comprises Fe₃O₄.

3. The magnetic nanoparticle according to claim 1, wherein the nanoporous silica shell is functionalized with equivalent amounts of primary and secondary amines.

4. The magnetic nanoparticle according to claim 1, wherein the nanoporous silica shell is functionalized with 2-aminoethyl-3-aminopropyl groups.

5. The magnetic nanoparticle according to claim 1, wherein the nanoporous silica shell is solid.

6. The magnetic nanoparticle according to claim 1, wherein the nanoporous silica shell is functionalized with a secondary amine.

7. The magnetic nanoparticle according to claim 1, wherein the nanoporous silica shell is functionalized with N-(trimethoxysilylpropyl)polyethylenimine.

8. The magnetic nanoparticle according to claim 1, wherein the nanoporous silica shell is hollow.

9. The magnetic nanoparticle according to claim 1, wherein the nanoporous silica shell has an amine concentration of 1 μmol per mg of magnetic nanoparticle or greater and the amine concentration is controlled by thickness of the nanoporous silica shell.

10. The magnetic nanoparticle according to claim 1, wherein the nanoporous silica shell has an amine concentration of 1-1.45 μmol per mg of magnetic nanoparticle when the nanoporous silica shell has a thickness of about 25 nm.

11. The magnetic nanoparticle according to claim 1, wherein the nanoporous silica shell has a thickness in a range of 2-100 nm.

12. The magnetic nanoparticle according to claim 1, wherein the nanoporous silica shell has a thickness in a range of 5-50 nm.

13. The magnetic nanoparticle according to claim 1, wherein the nanopores have an average size of 1-5 nm.

14. A process of producing an amine functionalized magnetic nanoparticle comprising: hydrolyzing tetraethoxysilane in a microemulsion of a superparamagnetic nanoparticle to form a superparamagnetic nanoparticle encapsulated by an incompletely hydrolyzed nanoporous silica shell having nanopores; and, hydrolyzing an amine-containing compound in situ in presence of the incompletely hydrolyzed nanoporous silica shell before hydrolysis and densification of the silica shell is complete to functionalize the nanoporous silica shell with amine groups both inside and outside the nanopores and to maintain nanoporosity of the shell.

15. The process according to claim 14, wherein the superparamagnetic nanoparticle comprises Fe₃O₄.

16. The process according to claim 14, wherein the superparamagnetic nanoparticle comprises a complex of Fe₃O₄ and oleic acid.

17. The process according to claim 14, wherein the amine-containing compound comprises a secondary amine.

18. The process according to claim 14, wherein the amine-containing compound comprises both a primary amine and a secondary amine.

19. The process according to claim 14, wherein the amine-containing compound comprises a hydrolysable group that can be hydrolyzed to facilitate bonding of the amine-containing compound to the silica shell.

20. The process according to claim 19, wherein the hydrolysable group is a silane group.

21. The process according to claim 14, wherein the amine-containing compound is N-(2-aminoethyl)-3-aminopropyltrimethoxysilane.

22. The process according to claim 14, wherein the amine-containing compound is with N-(trimethoxysilylpropyl)polyethylenimine.

23. The process according to claim 14, further comprising adsorbing a surfactant on to the surface of the superparamagnetic nanoparticle before hydrolyzing the tetraethoxysilane.

24. The process according to claim 23, wherein the surfactant is removed from the nanoporous silica shell after functionalization of the nanoporous silica shell with the amine groups.

25. The process according to claim 14, wherein hydrolyzing the amine-containing compound is carried out 8-30 hours after hydrolyzing tetraethoxysilane is begun.

26-31. (canceled)

32. A magnetic nanoparticle comprising: one or more cores comprising a superparamagnetic nanoparticle; and, a nanoporous silica shell surrounding the one or more cores, the shell having nanopores that extend to the surface of the magnetic nanoparticle, the shell functionalized with thiol groups both inside and outside the nanopores.

33. The magnetic nanoparticle according to claim 32, wherein the superparamagnetic nanoparticle comprises Fe₃O₄.

34. The magnetic nanoparticle according to claim 32, wherein the shell is functionalized with 3-mercaptopropyltrimethoxysilane.

35. The magnetic nanoparticle according to claim 32, wherein the shell is functionalized with S-nitrosothiol groups.

36-41. (canceled)
